(19) United States
(12) Patent Application Publication
(10) Pub. No.: US 2026/0000424 A1
(43) Pub. Date: Jan. 1, 2026

(54) TISSUE CUTTING ASSEMBLIES WITH AXIALLY MOVABLE BLADES

(71) Applicant: EDWARDS LIFESCIENCES CORPORATION, Irvine, CA (US)

(72) Inventors: Peleg Harel, Haifa (IL); Eitan Atias, Tel Aviv (IL); Noa Axelrod Manela, Kadima (IL); Ofir Witzman, Harish (IL)

(73) Assignee: EDWARDS LIFESCIENCES CORPORATION, Irvine, CA (US)

(21) Appl. No.: 19/317,928

(22) Filed: Sep. 3, 2025

Related U.S. Application Data
(63) Continuation of application No. PCT/US2024/019511, filed on Mar. 12, 2024.
(60) Provisional application No. 63/451,863, filed on Mar. 13, 2023.

(51) Int. Cl. A61B 17/3205; A61B 17/32; A61F 2/24

(52) U.S. Cl. CPC A61B 17/32053; A61F 2/2433; A61B 2017/320056

(57) ABSTRACT

The present disclosure relates to tissue cutting assemblies that can be used for cutting through a tissue. In an example, the tissue cutting assembly comprises a nosecone assembly and a cutting member extending therethrough. The nosecone assembly comprises a rigid member with a slot, and a flexible member positioned distally to, and attached to, the rigid member. The cutting member comprises a tubular body axially movable within a channel of the nosecone assembly, and a blade radially extending from the tubular body, configured to axially move between a concealed position, in which the blade is entirely concealed inside the slot, and an exposed position, in which a sharp edge of the blade is exposed from the nosecone assembly and can be further advanced to cut through the target tissue.

### CROSS-REFERENCE TO RELATED APPLICATIONS

This application is a continuation of International Application No. PCT/US2024/019511, filed Mar. 12, 2024, which claims the benefit of U.S. Provisional Application No. 63/451,863, filed Mar. 13, 2023, which is incorporated by reference herein.

### FIELD

The present disclosure relates to tissue cutting assemblies and delivery assemblies that can be used to form an opening in a target tissue, and to methods and devices for cutting through a target tissue that can be a leaflet of an existing valvular structure, in a manner that can modify existing valvular structures (for example, leaflets of a native heart valve or previously-implanted prosthetic valve) prior to implantation of a guest prosthetic valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (e.g., through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve (“guest valve”) is delivered to the heart in a crimped state, as described above for the “native” TAVR. The guest valve is positioned within the previously implanted prosthetic valve (“host valve”) and then expanded to its functional size. The host valve in a ViV TAVR procedure can be a surgically implanted prosthetic valve or a transcatheter prosthetic valve. The term “host valve” is also used herein to refer to the native aortic valve in a native TAVR procedure.

### SUMMARY

One known technique for mitigating the risk of coronary ostial obstruction involves lacerating or severing a portion of one or more leaflets of the host valve (which can be an aortic bioprosthetic valve or a native aortic valve). Lacerating or severing a portion of the leaflet(s) reduces the risk of blocking the coronary ostia when the guest prosthetic valve is implanted and displaces the leaflets of the host valve toward the inner wall of the aortic root. However, method that rely on lacerating existing leaflets, require high spatial precision and surgical skill. Moreover, once the leaflets have been lacerated, the existing heart valve may function poorly and increase the risk of aortic insufficiency, at least until a replacement prosthetic valve has been successfully implanted. If the existing leaflets have become calcified, there is a further risk that the lacerating will release particulate or other debris into the blood stream, which may make the patient susceptible to vascular occlusion or stroke.

According to some aspects of the disclosure, there is provided a tissue cutting assembly, comprising a nosecone assembly defining a nosecone channel. The nosecone assembly comprises a rigid member comprising at least one rigid member slot, a flexible member positioned distal to the rigid member and attached thereto and a cutting member. The cutting member comprises a tubular body extending through the nosecone channel, and at least one blade attached to the tubular body, the at least one blade comprising a sharp edge extending radially away from the tubular body. The cutting member is axially movable relative to the nosecone assembly between a concealed position and an exposed position, wherein the at least one blade is entirely concealed within the at least one rigid member slot when the cutting member is in the concealed position, and wherein at least a portion of the at least one blade is exposed out of the nosecone assembly when the cutting member is in the exposed position.

In one of its basic configurations, a delivery assembly comprises a guest prosthetic valve and a delivery apparatus comprising a tissue cutting assembly. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the guest prosthetic valve can comprise a frame movable between a radially compressed and a radially expanded configuration.

In some examples, the delivery apparatus can comprise a handle.

In some examples, the delivery apparatus can comprise a balloon catheter extending from the handle.

In some examples, the delivery apparatus can comprise a balloon mounted on the balloon catheter.

In some examples, the balloon catheter optionally defines a balloon catheter lumen.

In some examples, the balloon mounted on the balloon catheter is optionally in fluid communication with the balloon catheter lumen.

In some examples, the balloon is optionally configured to transition between deflated and inflated states thereof.

In some examples, the tissue cutting assembly can comprise a nosecone assembly optionally defining a nosecone channel.

In some examples, the tissue cutting assembly can comprise a cutting member.

In some examples, the nosecone assembly can comprise a rigid member optionally comprising at least one rigid member slot.

In some examples, the nosecone assembly can comprise a flexible member positioned distal to the rigid member and optionally attached thereto.

In some examples, the cutting member can comprise a tubular body extending through the nosecone channel.

In some examples, the cutting member can comprise at least one blade optionally attached to the tubular body.

In some examples, the at least one blade can comprise a sharp edge optionally extending radially away from the tubular body.

In some examples, the cutting member is optionally axially movable relative to the nosecone assembly, between a concealed position in which the at least one blade is entirely concealed within the at least one rigid member slot, and an exposed position in which at least a portion of the at least one blade is exposed out of the nosecone assembly.

In one of its basic methods, a method of forming an opening in a target tissue comprises advancing a tissue cutting assembly to a target tissue. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the tissue cutting assembly can comprise a nosecone assembly optionally defining a nosecone channel.

In some examples, the tissue cutting assembly optionally comprises a cutting member.

In some examples, the cutting member optionally comprises a tubular body optionally extending through the nosecone channel.

In some examples, the cutting member optionally comprises at least one blade with a sharp edge which is optionally concealed in at least one rigid member slot of a rigid member of the nosecone assembly, optionally proximal to a flexible member of the nosecone assembly which is optionally attached to the rigid member.

In some examples, the method comprises translating the cutting member distally relative to the nosecone assembly, so as to expose the sharp edge of the at least one blade.

In some examples, the method comprises forming a tissue opening within the target tissue by axially moving the at least one blade through the target tissue.

In one of its basic methods, a method of implanting a guest prosthetic valve within a host valvular structure comprises advancing a delivery assembly that comprises a delivery apparatus carrying a guest prosthetic valve in a radially compressed state, to a host valvular structure. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the delivery apparatus can comprise a balloon optionally mounted on a balloon catheter.

In some examples, the delivery apparatus can comprise a tissue cutting assembly.

In some examples, the tissue cutting assembly can comprise a nosecone assembly optionally defining a nosecone channel.

In some examples, the tissue cutting assembly can comprise a cutting member.

In some examples, cutting member can comprise a tubular body optionally extending through the nosecone channel.

In some examples, cutting member can comprise at least one blade with a sharp edge which is optionally concealed in a rigid member slot of a rigid member of the nosecone assembly, optionally proximal to a flexible member of the nosecone assembly which is optionally attached to the rigid member.

In some examples, the method comprises translating the cutting member distally relative to the nosecone assembly, so as to expose the sharp edge of the at least one blade.

In some examples, the method comprises forming a leaflet opening within a host leaflet of the host valvular structure by axially moving the at least one blade through the host leaflet.

In some examples, the method comprises positioning the balloon in a deflated state thereof, along with the guest prosthetic valve disposed in a compressed state over the balloon, inside the host valvular structure.

In some examples, the method optionally comprises inflating the balloon so as to radially expand the guest prosthetic valve.

The innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a cross-sectional view of a native aortic valve.

FIG. 2A shows a cross-sectional view of a prosthetic heart valve implanted in the native aortic valve of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

FIG. 4A shows a view in perspective of an exemplary tissue cutting assembly, in a concealed position of its cutting member.

FIG. 4B shows a view in perspective of the tissue cutting assembly of FIG. 4A, in an exposed position of its cutting member.

FIG. 5A shows a cross sectional view of the tissue cutting assembly in the concealed position of FIG. 4A.

FIG. 5B shows a cross sectional view of the tissue cutting assembly in the exposed position of FIG. 4B.

FIG. 6A shows an exploded view of unassembled components of an exemplary tissue cutting assembly.

FIG. 6B shows the tissue cutting assembly of FIG. 6A in an assembled configuration.

FIG. 7 shows an exemplary tissue cutting assembly having a flexible member slot.

FIG. 8 shows an exemplary tissue cutting assembly having a weakened line.

FIG. 9A shows an exploded view of unassembled components of an exemplary tissue cutting assembly comprising two blades.

FIG. 9B shows a view in perspective of the tissue cutting assembly of FIG. 9A in an assembled configuration.

FIG. 10A shows a cross sectional view of the tissue cutting assembly of FIG. 9B in a concealed position.

FIG. 10B shows a cross sectional view of the tissue cutting assembly of FIG. 9B in an exposed position.

FIG. 11A is a simplified side view of a tissue cutting assembly positioned with the cutting member in the concealed position within the nosecone assembly proximal to a host leaflet, according to an example.

FIG. 11B is a simplified side view of the tissue perforation cutting of FIG. 11A with the cutting member positioned in the host leaflet.

FIG. 11C is a simplified side view of the tissue perforation cutting of FIG. 11A with the cutting member positioned distal to the host leaflet.

FIG. 11D is a simplified side view of the tissue perforation assembly of FIG. 11A with the nosecone assembly passing through the host leaflet.

FIG. 12 shows a delivery assembly comprising a delivery apparatus that carries a prosthetic valve, and includes a tissue cutting assembly.

FIG. 13 is a cross-sectional view of the delivery apparatus of FIG. 12.

FIG. 14A shows the delivery assembly of FIG. 12 with guidewire positioned within a pilot puncture of the host leaflet.

FIG. 14B shows the delivery assembly of FIG. 12 with the blades of a cutting member passing through the host leaflet.

FIG. 15A is a simplified side view of the delivery assembly of FIG. 12 with a guest prosthetic valve crimped proximal to a deflated balloon which is positioned proximal to the leaflet opening.

FIG. 15B is a simplified side view of the delivery assembly of FIG. 15A with the guest prosthetic valve positioned over the deflated balloon, proximal to the leaflet opening.

FIG. 15C is a simplified side view of the delivery assembly of FIG. 15A with the compressed guest prosthetic valve and the deflated balloon positioned inside the leaflet opening of the host leaflet.

FIG. 15D is a simplified side view of the delivery assembly of FIG. 15A with the guest prosthetic valve expanded by the inflated balloon inside the host valvular structure.

FIG. 16 shows a previously implanted prosthetic valve subsequent to forming the leaflet opening in a host leaflet thereof.

FIG. 17 shows a configuration in which a guest prosthetic valve has been expanded within the leaflet opening of a host prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are devices and methods for implanting prosthetic valves and modifying leaflets of an existing valvular structure in a patient's heart. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve. The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices that can modify existing valvular structure, and in some implementations, implant prosthetic devices, to and/or in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

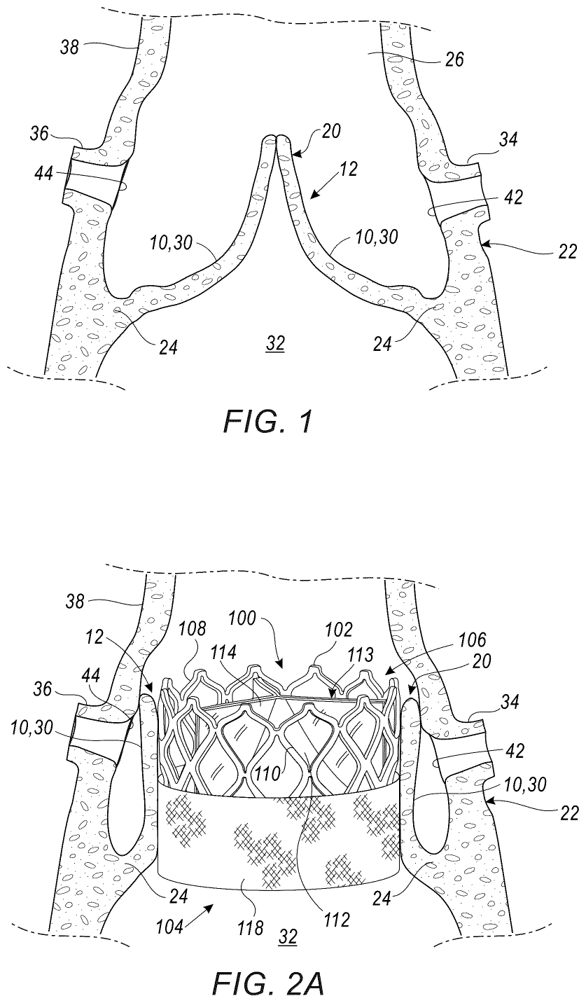

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures 40 (see FIG. 1B) by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

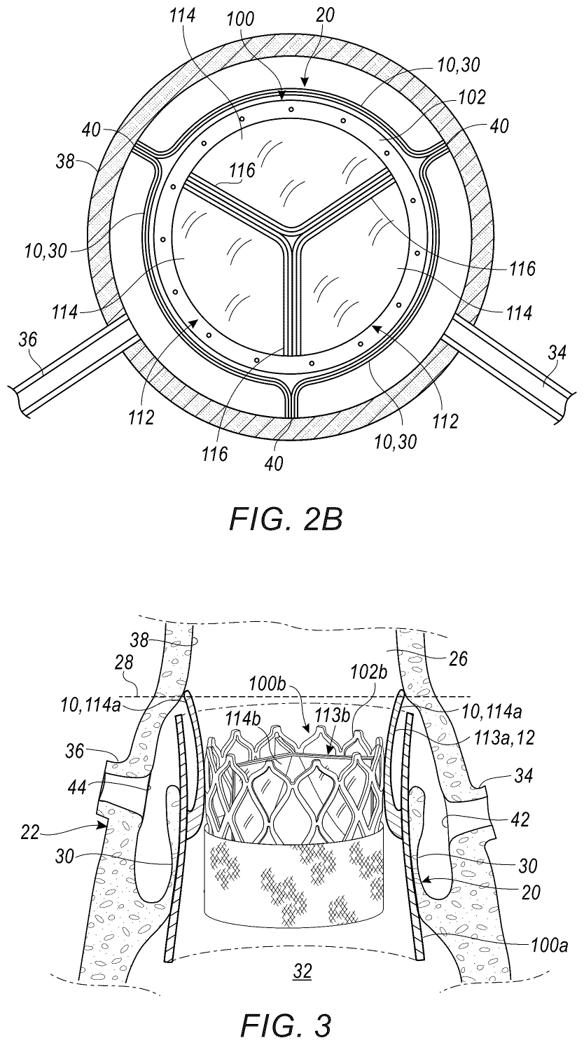

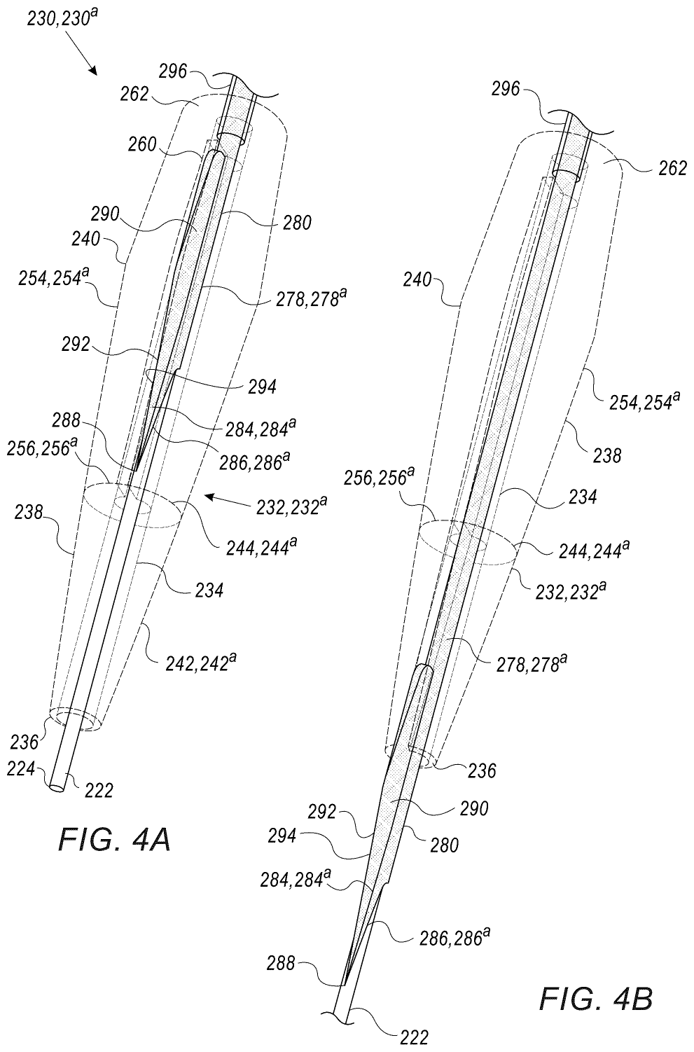

FIGS. 2A-2B show an exemplary prosthetic valve 100 that can be implanted in a native heart valve, such as the native aortic valve 20 of FIG. 1. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (such as delivery apparatus 202 described below with respect to FIG. 4, as well as other examples of delivery apparatuses described throughout the current disclosure) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/85,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can optionally be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can optionally be made of shape-memory materials such as, but not limited to, nickel titanium alloy (e.g., Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 can optionally be an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in U.S. Pat. No. 9,155,619, which is incorporated herein by reference.

The struts 108 can optionally include a plurality of angled struts and vertical or axial struts. At least some of the struts 108 can be pivotable or bendable relative to each other, so as to permit frame expansion or compression. For example, the frame 102 can optionally be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can optionally include a plurality of prosthetic valve leaflets 114 (e.g., three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can optionally be arranged together to form prosthetic valve commissures 116 that are coupled (directly or indirectly) to respective portions of the frame 102, thereby securing at least a portion of the valvular structure 113 to the frame 102. The prosthetic valve leaflets 114 can optionally be made from, in whole or part, biological material (e.g., pericardium), biocompatible synthetic materials, or other such materials. Further details regarding transcatheter prosthetic valves, including the manner in which leaflets 114 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can optionally comprise at least one skirt or sealing member. For example, the prosthetic valve 100 can optionally include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can optionally be sutured to the inner skirt along a scalloped line (not shown). An inner skirt can optionally be coupled to the frame 102 via sutures or another form of coupler.

The prosthetic valve 100 can optionally comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (e.g., PET) or natural tissue (e.g. pericardial tissue). In some cases, the inner skirt can optionally be formed of a single sheet of material that extends continuously around the inner surface of frame 102. In some cases, the outer skirt 118 can optionally be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (e.g., after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term “host valve” as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can optionally be a surgically implanted prosthetic heart valve (known as a “surgical valve”) or a transcatheter heart valve. The term “guest valve”, as used herein, refers to a prosthetic valve implanted in a host valve, which can optionally be either a native heart valve or a previously implanted prosthetic valve. Similarly, the term “host leaflets 10”, as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36. Any delivery apparatus described throughout the current disclosure, is advantageously configured to modify the host valvular structure 12 (i.e., modify at least one of the host leaflets 10), and optionally implant a guest prosthetic valve 100 within the modified valvular structure 12, optionally without the need to switch between separate delivery apparatuses for each function.

FIGS. 4A-10 show exemplary tissue cutting assemblies 230 that can optionally be used to cut through a tissue, such as a host leaflet 10 of a host valvular structure 12, so as to form an opening therein. Tissue cutting assembly 230 comprises a nosecone assembly 232 defining a nosecone channel 234, and a cutting member 278 having a tubular body 280 and at least one blade 290 axially movable through and relative to the nosecone assembly 232. The nosecone assembly 232 defines a distal tapering portion 238 extending from a narrower diameter of the nosecone assembly 232 at a nosecone distal edge 236, to a greater diameter of a tapering portion proximal end 240. The nosecone channel 234 defines an opening of the nosecone assembly 232 at the nosecone distal edge 236.

The nosecone assembly 232 comprises a flexible member 242 extending proximally from the nosecone distal edge 236 toward a flexible member proximal lip 244, and a rigid member 254 attached to the flexible member 242 and extending proximally therefrom. The rigid member 254 can optionally include a rigid member distal lip 256 that can be in contact with, and optionally coupled to, the flexible member proximal lip 244.

The outer surface of the flexible member 242 can optionally be flush and/or continuous with the outer surface of the rigid member 254. The nosecone distal edge 236 is the distal edge of the flexible member 242, and the tapering portion proximal end 240 is comprises in the rigid member 254, such that the outer surface of the flexible members 242, and a portion of the outer surface of the rigid member 254 extending between the rigid member distal lip 256 and the tapering portion proximal end 240, together form the distal tapering portion 238 of the nosecone assembly 232. The nosecone channel 234 extends in a continuous manner through both the rigid member 254 and the flexible member 242.

The rigid member 254 further comprises at least one rigid member slot 260, extending radially outwards from the nosecone channel 234 to the outer surface of the rigid member 254. The rigid member slot 260 proximally extends from the rigid member distal lip 256, and can optionally terminate, in some examples, at or proximate to the tapering portion proximal end 240. The rigid member 254 can optionally further include, in some examples, a rigid member proximal end portion 262 extending proximally from the rigid member slot 260, such that the rigid member proximal end portion 262 can serve as an unslotted portion of the rigid member 254.

The tubular body 280 of the cutting member 278 extends through at least a portion of the nosecone channel 234 and is axially movable, through the nosecone channel 234, relative to the nosecone assembly 232. In some examples, the tubular body 280 defines a cutting member lumen 282 sized to allow passage of a guidewire 222 therethrough. The guidewire 222 can optionally be axially movable, through the cutting member lumen 282, relative to the cutting member 278 and/or the nosecone assembly 232, and can optionally terminate at a guidewire tip 224.

The at least one blade 290 is attached to the tubular body 280, and includes a sharp edge 292 that can optionally extend radially outward in a diagonal manner along an angled edge portion 294. The tubular body 280 can optionally include a tube distal end portion 284, and the blade 290 can optionally be attached to, and extend radially away from, the tube distal end portion 284. In some examples, the at least one blade 290 is integrally formed with the tubular body 280.

The tube distal end portion 284 can optionally be a relatively sharp distal edge of the tubular body, configured to pierce through a target tissue, such as a host leaflet 12. In some examples, the tube distal end portion 284 comprises an angled surface 286 terminating at a tube tip 288, such that the tubular body 280 can optionally be in the form of a needle. The angled orientation, in the radial direction, of the angled surface 286 of tube distal end portion 284 can optionally be opposite to that of the angled edge portion 294 of the blade 290, such that the angled edge portion 294 shares the tube tip 288 of the angled surface 286, and forms an arrow-shaped cutting head together with the angled edge portion 294.

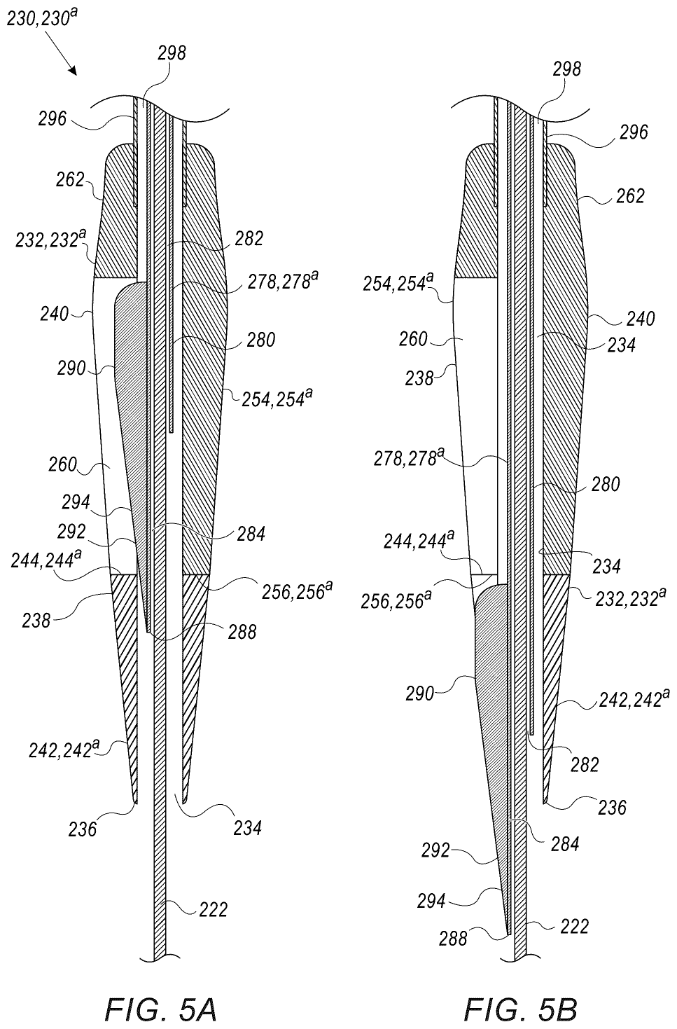

A nosecone shaft 296 defining a nosecone shaft lumen 298 can optionally be coupled to the nosecone assembly 232 and extend proximally therefrom. The nosecone channel 234 can optionally be continuous with the nosecone shaft lumen 298. The nosecone shaft 296 can optionally be coupled, directly or indirectly, to the rigid member 254. In some examples, a distal portion of the nosecone shaft 296 can optionally be coupled to rigid member proximal end portion 262, as illustrated in FIGS. 4A-5B. Attachment of rigid member 254 to a distal portion of nosecone shaft 296 can optionally be achieved by a variety of methods, such as overmolding, radio-frequency welding, through an adhesive, and/or a combination thereof. In some examples (not illustrated), the nosecone shaft 296 can optionally extend through the entire length of the nosecone channel 234, such that a distal end of the nosecone shaft 296 is aligned with the nosecone distal edge 236 and defines the distal opening of the nosecone assembly. In some examples (not illustrated), the nosecone shaft 296 is optionally coupled to one or more components, such as collars or other connectors, which are in turn attached to the rigid member 254.

The cutting member 278 is movable between a concealed position, in which the at least one blade 290 is entirely concealed within the nosecone assembly 232, such as within the rigid member slot 260 of rigid member 254, and an exposed position in which the sharp edge 292 of the blade 290 is exposed out of the nosecone assembly 232. FIGS. 4A and 4B show views in perspective of a tissue cutting assembly 230 in a concealed position and an exposed position, respectively, of the cutting member 278. The nosecone assembly 232 is shown in FIGS. 4A-4B with transparency to expose components extending therethrough. FIGS. 5A and 5B show cross-sectional views of the tissue cutting assembly 230 shown in FIGS. 4A and 4B, respectively.

Tissue cutting assembly 230 can optionally further include the guidewire 222 over which it can be advanced toward the site of treatment in a patient's body, such as a host valvular structure 12. In some examples, the guidewire 222 can be adapted to pierce through a target tissue, so as to form a pilot puncture in the target tissue, such as in a host leaflet 10. In some examples, the guidewire tip 224 can optionally be sharp enough to pierce through a target tissue when forcibly pushed there-against.

Various exemplary implementations for tissue cutting assemblies 230 can be referred to, throughout the specification, with superscripts, for ease of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, tissue cutting assembly 230^{a} is an exemplary implementation of tissue cutting assembly 230, and thus includes all of the features described for tissue cutting assembly 230 throughout the current disclosure, except that while a tissue cutting assembly 230 can include more than one blade 290 attached to the tubular body 280, tissue cutting assembly 230^{a} is shown to include a cutting member 278^{a} equipped with a single blade 290, as will be described in further detail below.

FIGS. 4A-5B show perspective and cross-sectional views of tissue cutting assembly 230^{a}. As mentioned above, tissue cutting assembly 230^{a} is an exemplary implementation of tissue cutting assembly 230, and thus includes all of the features described for tissue cutting assembly 230 throughout the current disclosure, except that a single blade 290 of the cutting member 278^{a} can be disposed in, and axially move through, a single rigid member slot 260 of the rigid member 254^{a}.

In some examples, the flexible member proximal lip 244^{a} can optionally extend radially outwards from the nosecone channel 234, and the rigid member distal lip 256^{a} can optionally similarly extend radially outwards from the nosecone channels 234. The flexible member proximal lip 244 can optionally be attached to the rigid member distal lip 256, such as, but not limited to, gluing, RF welding, and the like.

As illustrated in FIGS. 4A and 5A, the rigid member slot 260 is sized such that the blade 290 can optionally be entirely concealed therein in the concealed position. For example, the radial profile and dimensions of the rigid member 254 can optionally be designed such that in the concealed position, the sharp edge 292 does not extend radially outward away from the rigid member slot 260.

Since the distal tapering portion 238 narrows in diameter in the distal direction, advancement of the cutting member 278 relative to the nosecone assembly 232, such as by distally pushing the tubular body 280 relative to the nosecone assembly 232, and/or proximally pulling the nosecone assembly 232 relative to the cutting member 278, serves to expose the sharp edge 292 of blade 290 beyond the narrower portions of the distal tapering portion 238, and optionally exposing the whole of the tube distal end portion 284 and/o blade 290 out of nosecone assembly 232, such that the tube distal end portion 284 and/o blade 290 are positioned distally to the nosecone distal edge 236. It is to be understood that as the blade 290 is advanced through the nosecone assembly 232, the blade 290 can be gradually exposed from the flexible member 242, such that a plurality of partially exposed positions of the cutting member 278 can be assumed during such advancement, and a completely exposed position of the cutting member 278 can be achieved when the whole of the sharp edge 292 is exposed, and optionally such that at least the tube tip 288 and/or the whole of the tube distal end portion 284 is distal to the nosecone distal edge 236.

To protect the patient's anatomy from the sharp edge 292 of the blade 290 prior to forming an opening through a target tissue, the cutting member 278 is retained in the concealed position during delivery of the tissue cutting assembly 230 through the patient's vasculature. The rigid member 254 is formed of a relatively stiff material, which is sufficient to provide structural support to the sidewalls of rigid member slot 260, such that the rigid member slot 260 can adequately support the blade 290 accommodated therein during delivery of the tissue cutting assembly 230 through the patient's vasculature, to protect the sharp edge 292 from inadvertently contacting native tissue other than the target tissue. The rigid member proximal unslotted end portion 262 can prevent the blade 290 from sliding to a position proximal to the nosecone assembly 232.

The flexible member 242 can optionally be relatively more flexible than the rigid member 254, to assist in advancement of the nosecone assembly 232 through the patient's vasculature. Flexibility of the flexible member 242 can assist in navigating around a bent or curve in the patient's vasculature. Increased flexibility of distal flexible member 242 allows a certain length thereof to get around a bend in a patient's vasculature, and create a “follow-the-leader” effect with the remainder of the nosecone assembly 232, including rigid member 254.

In some implementation, rigid member 254 is stiffer than flexible member 242. In some examples, rigid member 254 is made from a material which is stiffer than that of flexible member 242. Flexible member 242 can optionally be made of a relatively soft polymeric material, such as a material having a hardness in the range from about 50 Shore A to 80 Shore D, and in some examples, in the range from about 60 Shore A to 100 Shore A. Suitable polymeric materials include polyurethane, polyester, polyvinyl chloride, polyethylene, polyamide (nylon), and the like. In contrast, the rigid member 254 can be formed of a material having a higher hardness, such as, but not limited to, higher than 80 Shore D. Suitable materials can include polymeric materials and/or metallic materials.

FIGS. 4A-5B show an exemplary flexible member 242^{a} which does not include a slot when provided, prior to insertion into the patient's vasculature and/or reaching the target tissue. The flexible member 242^{a} can optionally be formed as full-matter surrounding the entire circumference around the nosecone channel 234, which can provide the required integrity during advancement through the patient's vasculature toward the target tissue. Advancement of the cutting member 278 relative to the nosecone assembly 232, from the concealed position shown in FIGS. 4A and 5A, will force the sharp edge 292 to engage with the flexible member 242 when the blade 290 is pushed past the rigid member slot 260. The material from which the flexible member 242 is formed can be soft enough such that the blade 290 can cut through the flexible member 242 when advanced in a distal direction therethrough, which in turn forms an axial cut through the flexible member 242 which is continuous with the rigid member slot 260, as shown in FIGS. 4B and 5B.

Thus, the material from which the rigid member 254 is formed can be stiff enough to resist cutting thereof by the blade 290, allowing axial passage of the blade 290 only through a pre-formed rigid member slot 260, while the flexible member 242 can optionally be formed of a material that can be cut by the blade 290, allowing it to optionally be devoid of a pre-formed slot, and to allow passage of the blade 290 therethrough by forcibly pushing the blade 290 in a manner that cuts through the flexible member 242.

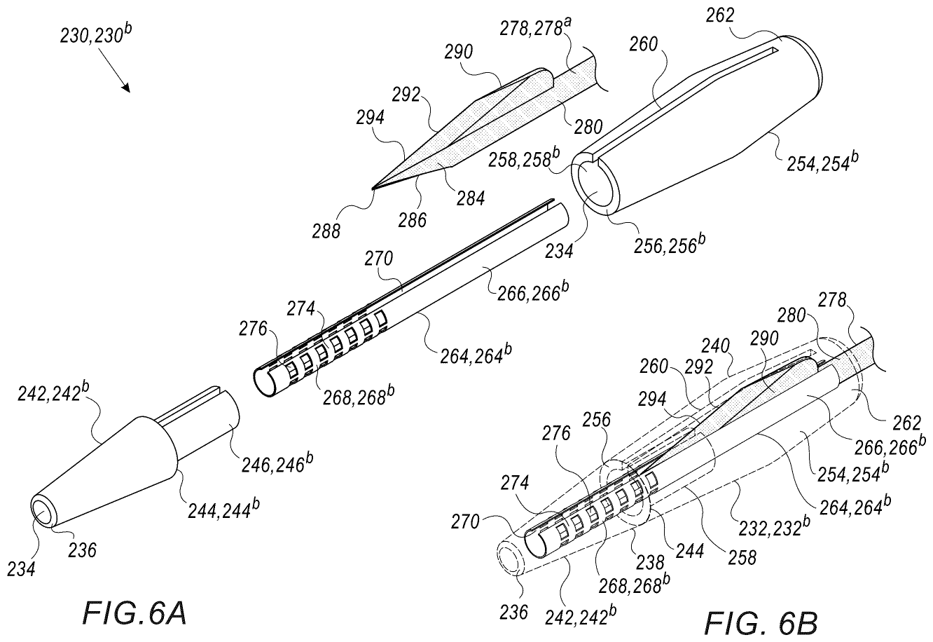

FIG. 6A shows an exploded view of unassembled components of an exemplary tissue cutting assembly 230^{b}. FIG. 6B shows the tissue cutting assembly 230^{b} of FIG. 6A in an assembled configuration. Tissue cutting assembly 230^{b} is an exemplary implementation of tissue cutting assembly 230, and thus includes all of the features described for tissue cutting assembly 230 throughout the current disclosure, except that the cutting assembly 230^{b} further comprises at least one support member 264. A support member 264 can be disposed within the nosecone assembly 232, extending axially along at least a portion of the nosecone channel 234. The support member 264 is disposed between the nosecone assembly 232, such as the outer surface of nosecone channel 234, and the cutting member 278, to provide additional structural support to the cutting member 278 inside the nosecone assembly 232. The nosecone assembly 232^{b} is shown with transparency in FIG. 6B to exposed components disposed therein.

The support member 264 can optionally comprise a support member proximal portion 266, disposed within the rigid member 254, and a support member distal portion 268, disposed within the flexible member 254. The support member distal portion 268 can optionally include a plurality of openings 276 separated by ribs 274, which can increase flexibility of the support member distal portion 268 inside the flexible member 254. The support member proximal portion 266 can optionally be devoid of opening 276, as it can be more rigid and does not need to flex or bend inside the rigid member 254.

In some examples, a single support member 264^{b} can optionally include a support member slot 270 extending along its length, and the support member 264^{b} can be formed as a C-shaped member extending, in cross-section, around the entire perimeter of the nosecone channel 234 between both sides of the support member slot 270. The support member slot 270 is aligned with the rigid member slot 260, to allow uninterrupted axial passage of the blade 290 along the support member 264^{b}.

In some examples, the flexible member 242 can optionally include a proximal extension 246 extending proximally from the flexible member proximal lip 244, and the rigid member 254 can optionally include a socket 258 configured to accommodate the proximal extension 246 therein. The proximal extension 246 and socket 258 can optionally be attached to each other by a variety of methods, such as overmolding, radio-frequency welding, through an adhesive, and/or a combination thereof. Advantageously, the proximal extension 246^{b} and socket 258^{b}, illustrated in FIGS. 6A-6B, can optionally provide greater surface area for bonding, when compared to flat planar lips 244^{a}, 256^{a} devoid of such a proximal extension and socket, as illustrated in FIG. 4A-5B. In some examples, the proximal extension 246^{b} and/or socket 258^{b} can optionally be tubular in shape, and can optionally include slots that can be aligned with rigid member slot 260 and/or support member slot 270.

While proximal extension 246 and socket 258 are shown in combination with a tissue cutting assembly 230^{b} that includes a support member 264, it is to be understood that this is not meant to be limiting, and that any tissue cutting assembly 230, including tissue assembly 230^{a} illustrated without a support member, can optionally include a flexible member 242 provided with a proximal extension 246, and a rigid member 254 provided with a complementary-shaped socket 258.

FIG. 7 shows a view in perspective of a tissue cutting assembly 230^{c}. Tissue cutting assembly 230^{c} is an exemplary implementation of tissue cutting assembly 230, and thus includes all of the features described for tissue cutting assembly 230 throughout the current disclosure, except that the flexible member 242^{c} of cutting assembly 230^{c} further comprises at least one flexible member slot 248. Thus, in some examples, and in contrast to the full-matter illustration of a flexible member 242 shown in FIGS. 4A-5B, the flexible member 242^{c} can optionally include a pre-formed flexible member slot 248 extending radially away from the nosecone channel 234. The flexible member slot 248 is continuous with the rigid member slot 260, and can optionally extend all the way to the nosecone distal edge 236. In such examples, the blade 290 does not need to cut through the material of the flexible member 242^{c} as it is advanced from the concealed position to the exposed position. This allows the flexible member 242^{c} to be formed from a material which is more flexible than that of the rigid member 254, yet not necessarily soft enough to be easily cut by the blade 290.

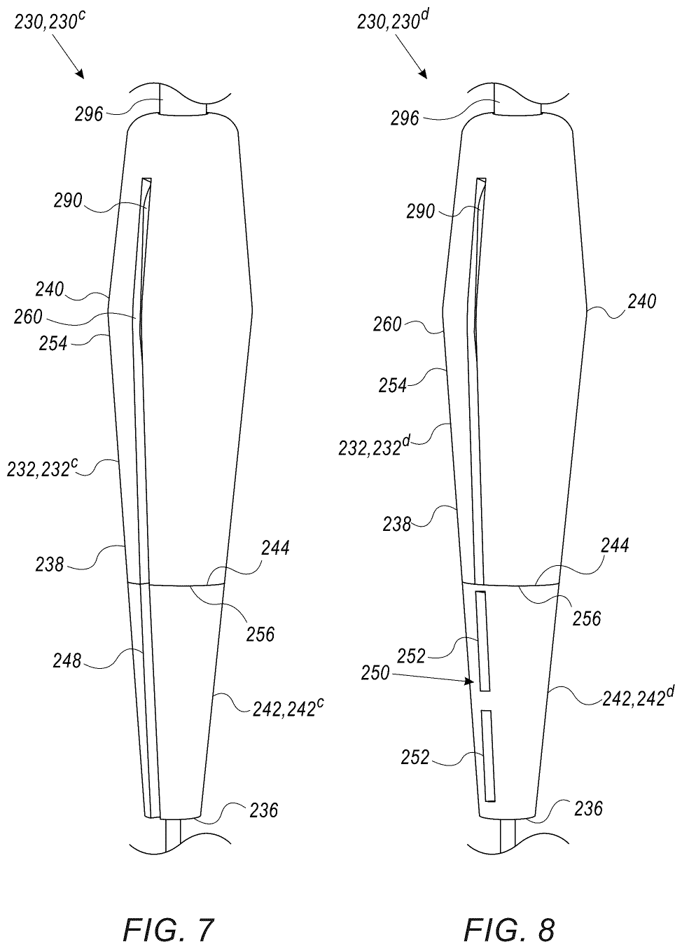

FIG. 8 shows a view in perspective of a tissue cutting assembly 230^{d}. Tissue cutting assembly 230^{d} is an exemplary implementation of tissue cutting assembly 230, and thus includes all of the features described for tissue cutting assembly 230 throughout the current disclosure, except that the flexible member 242^{d} of cutting assembly 230^{d} further comprises at least one weakened line 250. A weakened line 250 can be configured to allow easier cutting of the flexible member 242^{c} as the blade 290 is axially advanced therethrough, from the concealed position to the exposed position. In some examples, the weakened line 250 can optionally comprise one or more slits 252 as shown in FIG. 8. In some examples, the weakened line 250 can optionally be in the form of a score-line, or can optionally be a thinned lined having a smaller thickness relative to other portions of the flexible member 242.

It is to be understood that any exemplary tissue cutting assemblies 230 disclosed herein, including any example of tissue cutting assembly 230^{a} or of tissue cutting assembly 230^{b} described above, can optionally include a flexible member slot 248 as described for tissue cutting assembly 230^{c} with respect to FIG. 7, or a weakened line 250 as described for tissue cutting assembly 230^{d} with respect to FIG. 8.

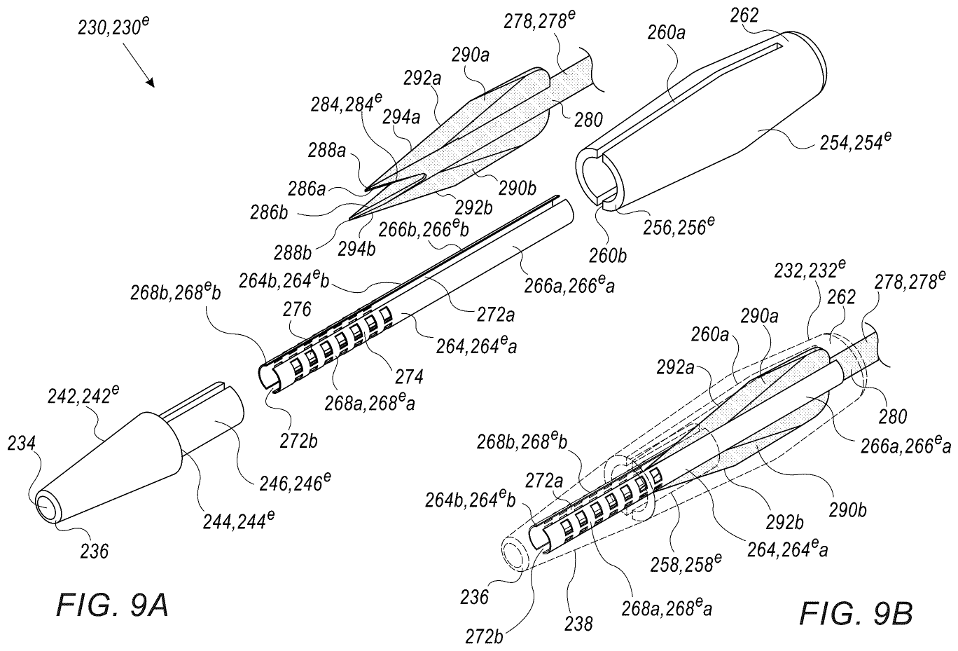

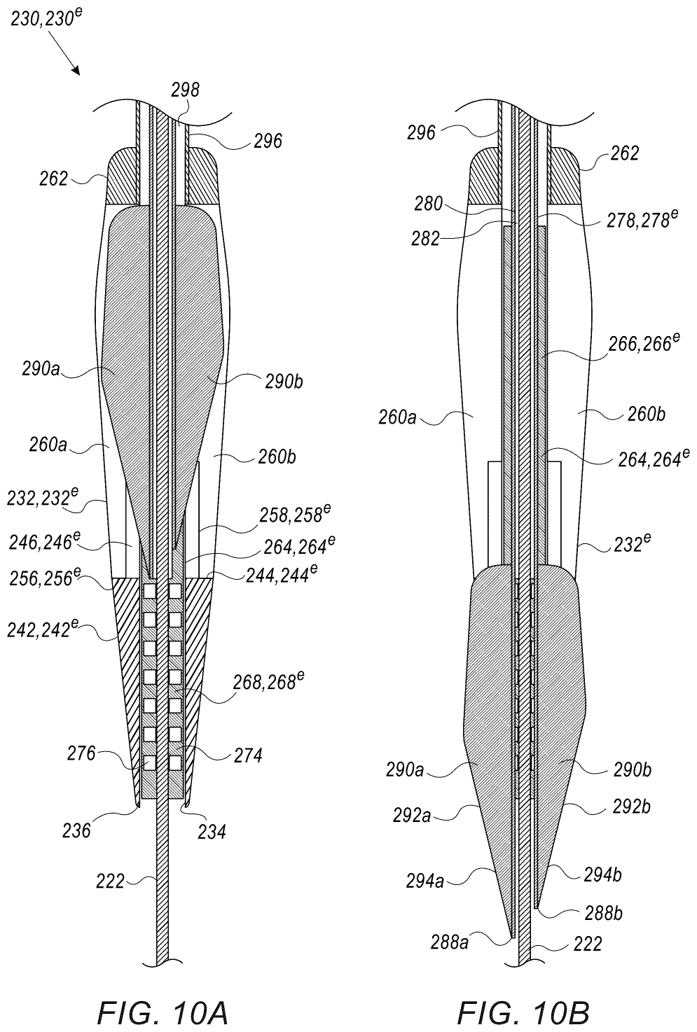

FIG. 9A shows an exploded view of unassembled components of an exemplary tissue cutting assembly 230^{e}. FIG. 9B shows the tissue cutting assembly 230^{e} of FIG. 9A in an assembled configuration. FIGS. 10A and 10B show cross-sectional views of the tissue cutting assembly 230^{e} of FIG. 9B, in a concealed position and an exposed position, respectively, of the cutting member 278^{e}. Tissue cutting assembly 230^{e} is an exemplary implementation of tissue cutting assembly 230, and thus includes all of the features described for tissue cutting assembly 230 throughout the current disclosure, except that the cutting member 278^{e} includes two blades 290a and 290b, and the rigid member 254^{e} includes two rigid member slots 260a, 260b through which the blades 290a, 290b can optionally extend.

Each of the two blades 290a and 290b can optionally radially extend from the tubular body 280 in an opposite direction. In some examples, the tube distal end portion 284^{e} can optionally include two angled surfaces 286a and 286b, terminating at corresponding tube tips 288a and 288b, wherein both angled surfaces 286a and 286b can optionally converge at the opposite ends to tips 288a, 288b, forming together a U-shaped or V-shaped angled edge portion. The sharp edge 292b of blade 290a can optionally extend along an angled edge portion 294a, sharing the tube tip 288a, and the sharp edge 292b of blade 290b can optionally extend along an angled edge portion 294b, sharing the tube tip 288b. In some examples, the tube tips 288a and 288b are axially offset from each other. In the illustrated examples, tube tip 288b can optionally be a distal tube tip, from which a distal blade 290b extends, and tube tip 288a can optionally be a proximal tube tip, from which a proximal blade 290a extends. This configuration can allow gradual cutting or perforation of the target tissue, such that when the tube distal end portion 284^{e} is advanced toward and through the target tissue, the distal tube tip 288b and distal blade 290b first pierce through the target tissue, followed by the proximal tube tip 288a and proximal blade 290a that subsequently pierce through the tissue in a gradual manner.

Optionally, the slots 260a and 260b can radially extend in opposite directions from the nosecone channel 234, in alignment with the blades 290a and 290b, respectively. In some examples, at least one support members comprises two support members 264^{e}a and 264^{a}b, which can optionally be C-shaped support members 264^{e} separated from each other by two gaps 272a, 272b. The gaps 272a, 272b can optionally be aligned with the rigid member slots 260a, 260b. When provided with a proximal extension 246^{e} and a corresponding socket 258^{e}, each can optionally be provided with two opposing slots that can optionally be aligned with the rigid member slots 260a. 260b and/or the gaps 272a, 272b.

While illustrated to include two support members 264^{e}, it is to be understood that this is shown by way of illustration and not limitation, and that a tissue cutting assembly 230^{e} can optionally be provided without support members, in similar manner to that described above with respect to tissue cutting assembly 230^{a}. While illustrated to include a proximal extension 246^{e} and socket 258^{e}, it is to be understood that this is shown by way of illustration and not limitation, and that a tissue cutting assembly 230^{e} can optionally be provided without proximal extension and socket, in similar manner to that described above with respect to tissue cutting assembly 230^{a}.

While the flexible member 242^{e} is shown to be provided as full-matter flexible member, devoid of pre-formed slots or weakened lines, it is to be understood that this is shown by way of illustration and not limitation, and that a flexible member 242^{e} of tissue cutting assembly 230^{e} can optionally be provided with at least one flexible member slot 248, in similar manner to that described above with respect to tissue cutting assembly 230^{c}, or with at least one weakened line 250, in similar manner to that described above with respect to tissue cutting assembly 230^{d}. When provided with at least one pre-formed flexible members slot 248, flexible member 242^{e} can optionally include two flexible members slots 248 which are continuous with the two rigid member slots 260. When provided with at least one weakened line 250, flexible member 242^{e} can optionally include two weakened lines 250 which are in alignment with the two rigid member slots 260.

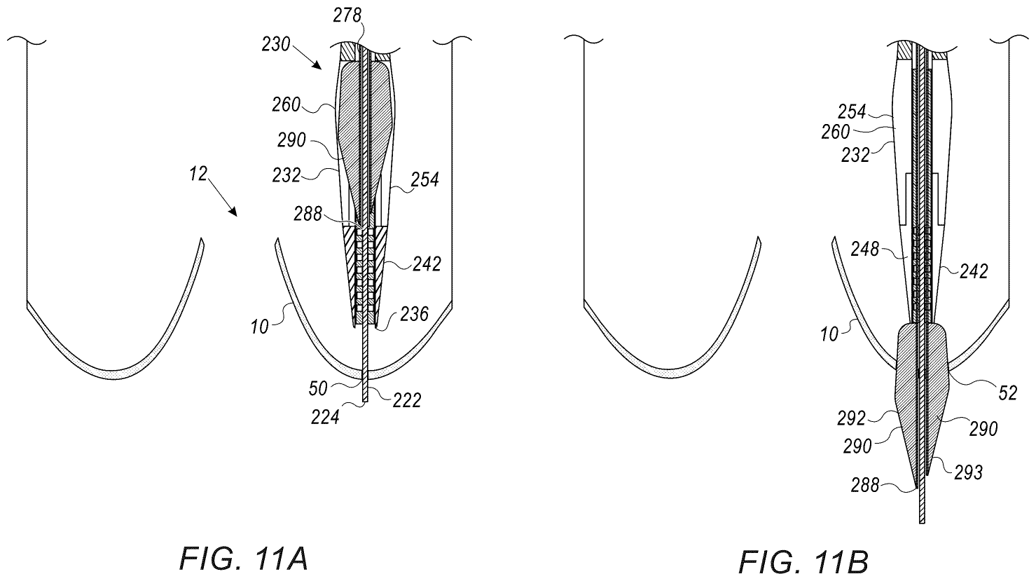

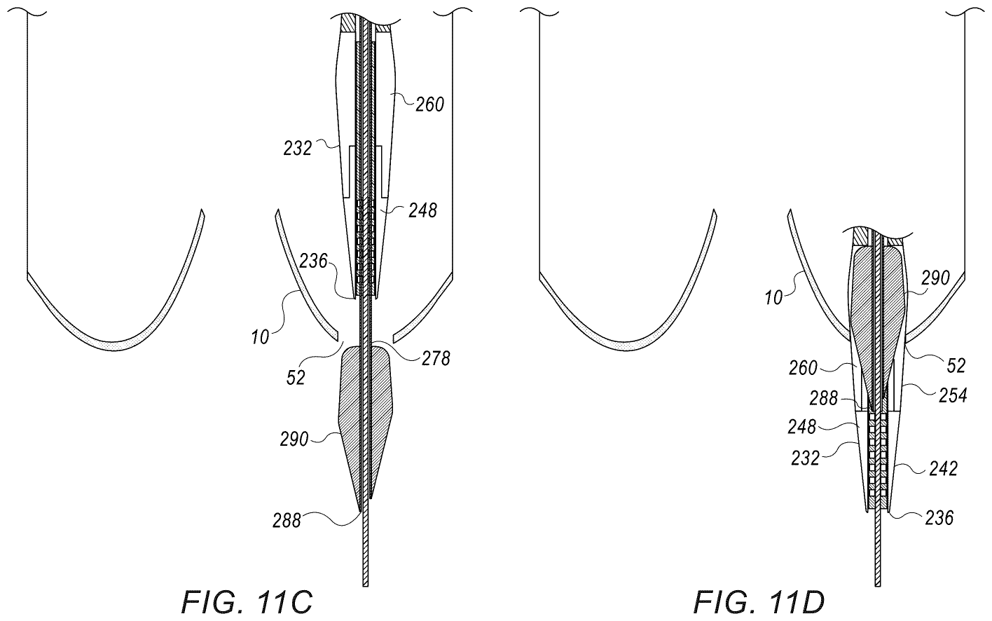

FIGS. 11A-11D illustrate some steps in a method for utilizing a tissue cutting assembly 230 for forming an opening within a target tissue. An exemplary implementation of the method is illustrated in FIGS. 11A-11D with respect to forming a leaflet hole inside a host leaflet, which can optionally be performed prior to implanting a guest prosthetic valve inside the host valvular structure. The tissue cutting assembly 230 can optionally be used to perforate, cut, and/or tear a host leaflet 10, such as a native leaflet 30 or a prosthetic valve leaflet 114 of a previously implanted prosthetic valve. While tissue cutting assembly 230^{e} is illustrated throughout FIGS. 11A-11D, it is to be understood that other implementations of tissue cutting assemblies 230 described in the current specification can optionally be used in a similar manner.

The distal end portion of the tissue cutting assembly 230, including nosecone assembly 232, is configured to be inserted into a patient's vasculature, such as within an ascending aorta, and to be advanced towards the host leaflet 10, wherein the guidewire 222 can optionally pierce through the host leaflet 10 as shown in FIG. 11A. Positioning tissue cutting assembly 230 relative to the host leaflet 10 may optionally comprise advancing the tissue cutting assembly 230 toward the leaflet via guidewire 222. Delivery of the tissue cutting assembly 230 toward the target tissue, such as host leaflet 10 of host valvular structure 12, is performed such that the cutting member 278 is retained in a concealed position, to protect the surrounding tissues from being contacted by its sharp edge during delivery.

As mentioned above, the cutting member 278 is configured to be selectively translated in the proximal or distal directions relative to nosecone assembly 232. Thus, distal advancement of the cutting member 278 from the concealed position to the exposed position, serves to expose the sharp edge(s) 292 of the blade(s) 290 from the nosecone assembly 232, optionally cutting through the flexible member 242 during distal advancement of the cutting member 278, as described above. As shown in FIG. 11B, the cutting member 278 is configured to puncture and/or cut through the host leaflet 10 to form an opening 52 within host leaflet 10 when passed through the host leaflet 10 in the exposed position of the cutting member 278.

As further mentioned above, the tubular body 280 can optionally comprise a cutting member lumen 282, configured to accommodate a guidewire 222 that can optionally extend through the cutting member lumen 282. The guidewire 222 can optionally be inserted into the patient's vasculature, and then tissue cutting assembly 230 may be advanced toward the host leaflet 10 over the guidewire 222.

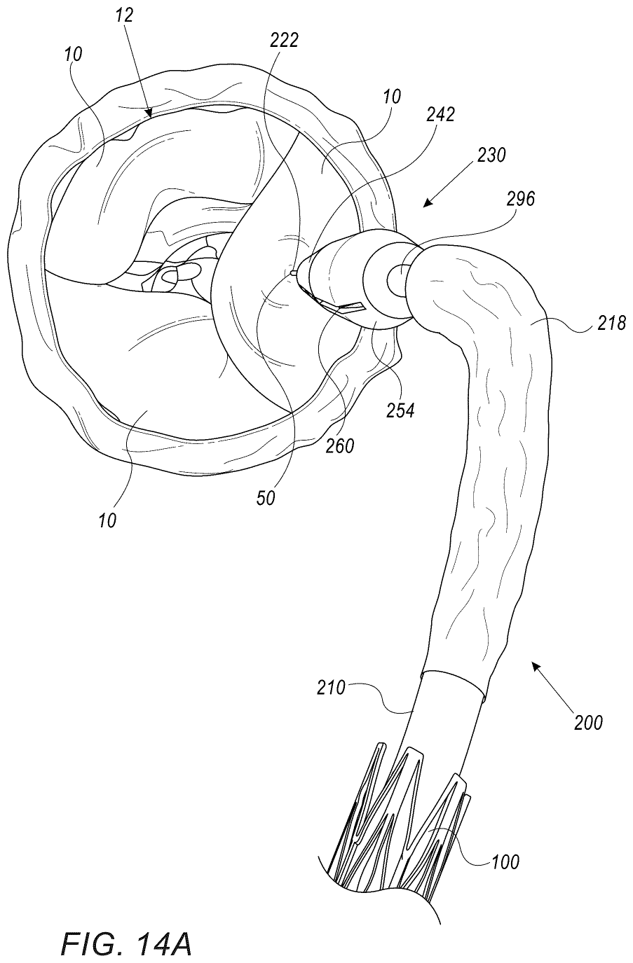

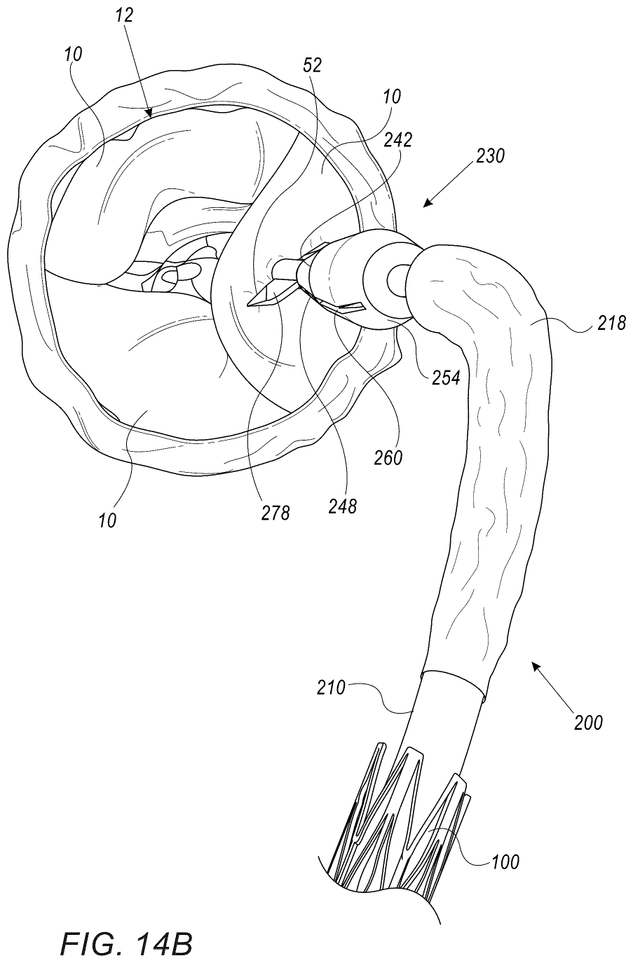

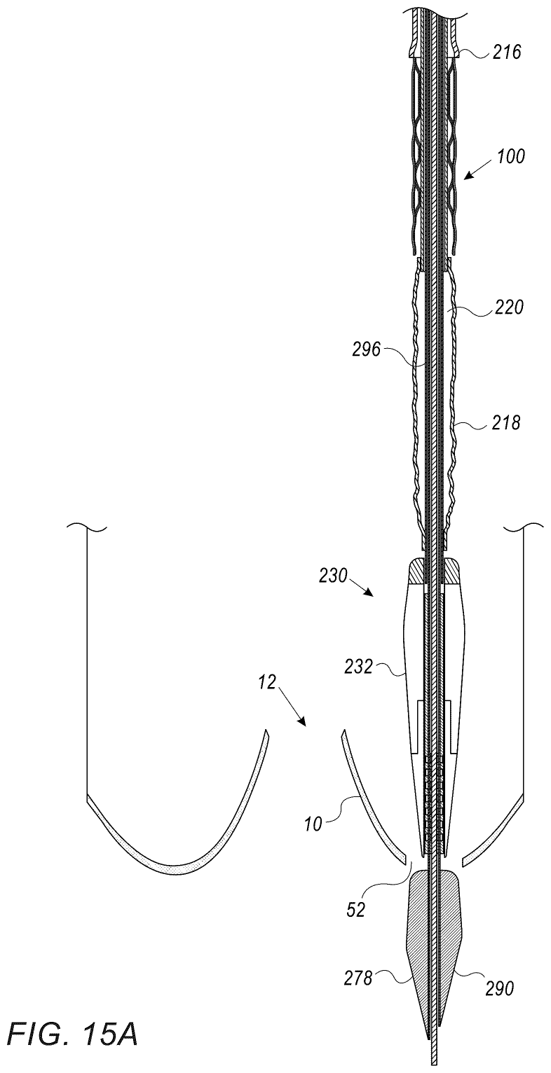

In some examples, the guidewire 222 can optionally be used as a perforating or lacerating member for forming a pilot puncture 50 (shown in FIG. 14A, for example). In such examples, the guidewire 222 can optionally be a relatively stiff wire having a distal tip 224 configured to pierce the host leaflet 10 when the guidewire 222 is pressed against the leaflet. In some examples, the guidewire 222 can optionally include a radio-frequency (RF) energy delivery tip 224 to assist with penetration through the leaflet tissue. For this purpose, a suitable RF energy device may optionally be coupled to the guidewire 222, and the RF energy device can apply the RF energy to the guidewire tip 224 to penetrate the host leaflet 10. In any examples disclosed herein wherein a guidewire is used to puncture a target tissue, such as a host leaflet, the guidewire 222 can optionally be coupled to a source of RF energy that applies RF energy to the tip of the guidewire. When the guidewire 222 is used to pierce the leaflet 10, the tube distal end portion 284 can be devoid of an angled surface 286, or it can also include an angled surface 286 used in combination with the guidewire 222 to form an initial puncture in the leaflet 10, wherein continued advancement of the cutting member 278 expands the puncture 50 to cut through the tissue and form a wider tissue opening 52.

In some examples, the guidewire tip 224 is not necessarily sharp enough or otherwise configured to puncture through the host leaflet 10, in which case the guidewire 222 can optionally be utilized for advancement of the tissue cutting assembly 230 toward the valvular structure 12, but terminate in proximity of the host leaflet 10 without piercing through it (for example, remaining above host leaflet 10 instead of passing through the tissue as shown in FIG. 11A), and the cutting member 278, after being moved from the concealed position to the exposed position, can optionally be then advanced toward and into the host leaflet 10, to form the leaflet opening 52 in a similar manner to that illustrated in FIG. 11B.

After formation of the leaflet opening 52, as shown in FIG. 11C, the nosecone assembly 232 can optionally be distally passed through the leaflet opening 52, as shown in FIG. 11D. In some examples, when the radius of the tapering portion proximal end 240 is greater than the radial distance of the blade(s) 290 from the central axis of the tubular body 280, the nosecone assembly 232 can serve as a dilator that further expands the leaflet opening 52. In some examples, the blade(s) 290 can optionally be kept in position, such as below the host leaflet 10 as shown in FIG. 11C, while the nosecone assembly 232 is advanced through the leaflet opening 52, until the nosecone assembly 232 fully covers the blade(s) 290, reverting the cutting member 278 to the concealed position, as shown in FIG. 11D. In some examples, the cutting member 278 can optionally be retracted proximally back into the concealed position inside the nosecone assembly 232 after formation of the leaflet opening 52, and prior to advancement of the nosecone assembly 232 through the leaflet opening 52.

In some examples, passing components of the tissue cutting assembly 230, such as cutting member 278 and/or nosecone assembly 232, through the host leaflet 10, may cause the host leaflet 10 to rip and/or tear such that the leaflet opening 52 is not a bounded hole. Stated differently, in such examples, the leaflet opening 52 may be formed by a tear that extends to the free edge of the host leaflet 10 (the coaptation edge of the leaflet).

The tissue cutting assembly 230 may be configured to form the leaflet opening 52 in any of a variety of host valvular structures 12. In the example of FIGS. 11A-11D, the host valvular structure 12 can be the valvular structure 113 of a previously implanted prosthetic valve, such as the prosthetic valve 100a of FIG. 3. In such examples, using the tissue cutting assembly 230 as described herein to form the leaflet opening 52 in a previously implanted prosthetic valve may optionally be followed by steps for implanting a guest prosthetic valve 100b within the previously implanted prosthetic valve 100a (for example, via a ViV procedure).

Similarly, the host valvular structure 12 in the example of FIGS. 11A-11D can optionally be a valvular structure 29 of a native heart valve, such as the native aortic valve 20 shown in FIGS. 2A-2B. In such examples, the tissue cutting assembly 230 can be configured to puncture a native leaflet 30 of the native aortic valve 20. In other examples, the host valvular structure and/or the native valve may refer to another valve of a patient's heart, such as a mitral valve, a pulmonary valve, or a tricuspid valve.

While illustrated and described above with respect to forming a leaflet opening 52 within a host leaflet 10, it is to be understood that the tissue cutting assembly 230 may be configured to form a tissue opening through other tissues in a patient's body. For example, prosthetic devices can be delivered to the left atrium or the left ventricle in a transseptal approach, wherein a delivery apparatus is passed through the vena cava, into the right atrium, and through the interatrial septum tissue. Such delivery approaches require puncturing the interatrial septum. Thus, in some implementations, a tissue cutting assembly 230 may optionally be utilized to form an opening through the interatrial septum, for example at the site of the fossa ovalis, which is a region of the septum containing tissue of lesser thickness than is typical of the rest of the septum. Thus, any example of tissue cutting assembly 230 described above can optionally be utilized in a manner similar to that described with respect to FIGS. 11A-11D, to form a tissue opening, equivalent to leaflet opening 52 described with respect to FIG. 11A-11D, in a target tissue, equivalent to a host leaflet 10 described with respect to FIGS. 11A-11D.

In some examples, tissue cutting assembly 230 can optionally be part of a delivery assembly that includes a delivery apparatus carrying a prosthetic valve. Any delivery assembly disclosed herein, comprises a delivery apparatus that can include a tissue perforation assembly according to any of the examples described above, and a balloon expandable prosthetic valve. While examples of a delivery assembly described in the current disclosure, are shown to include an exemplary delivery apparatus and a balloon expandable prosthetic valve, it should be understood that a delivery apparatus that includes a tissue cutting assembly according to any example of the current disclosure can optionally be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

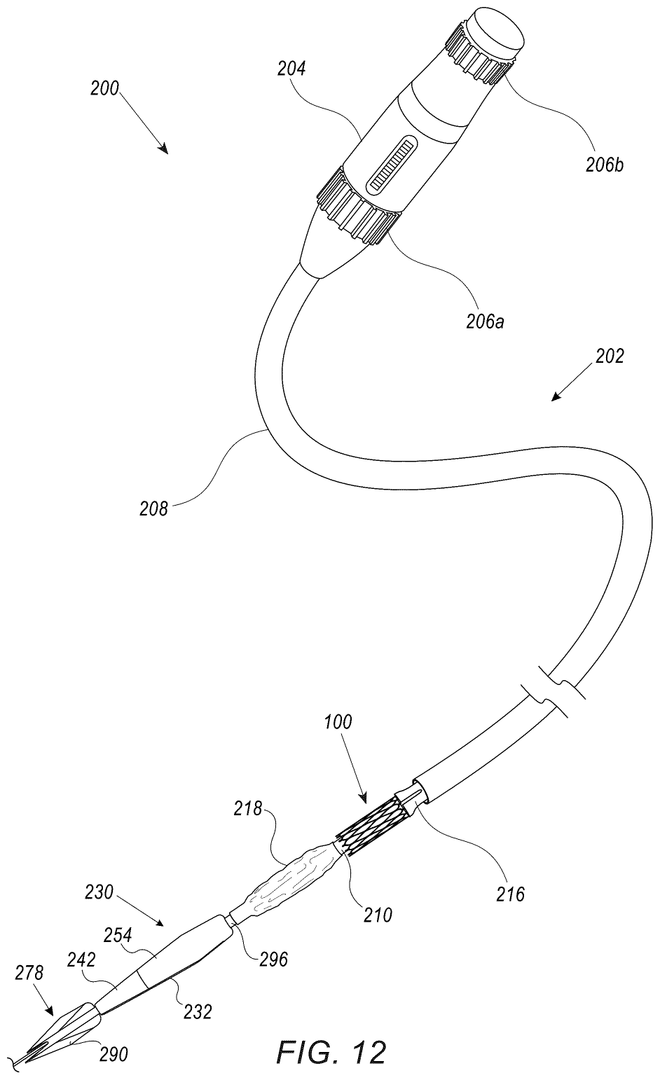

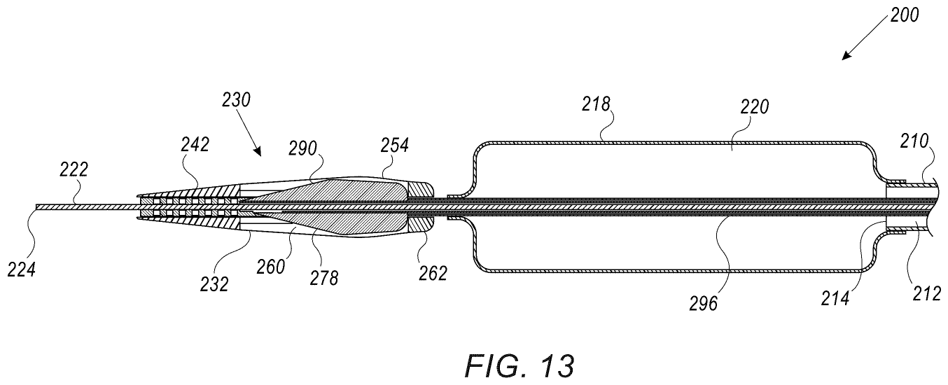

FIG. 12 illustrates an exemplary delivery assembly 200 that includes an exemplary delivery apparatus 202 adapted to deliver a balloon expandable prosthetic valve 100, such as prosthetic valve 100 described above with respect to FIGS. 2A-2B. FIG. 13 shows a cross-sectional view of a distal portion of delivery apparatus 202 comprising a tissue cutting assembly 230. It is to be understood that a tissue cutting assembly 230^{e} equipped with a cutting member 278^{e} having two blades 290 is shown in FIGS. 12-13 by way of illustration and not limitation, and that the delivery apparatus 202 includes a tissue cutting assembly 230 which can optionally be implemented according to any of the examples described above. According to some examples, the delivery apparatus 202 further includes a handle 204 and a balloon catheter 210 having a balloon 218 mounted on its distal end, proximal to the nosecone assembly 232. The balloon expandable prosthetic valve 100 can optionally be carried in a crimped state over the balloon catheter 210. Optionally, an outer delivery shaft 208 can concentrically extend over the balloon catheter 210, and a push shaft 216 can be disposed over the balloon catheter 210, optionally between the balloon catheter 210 and the delivery shaft 208. The nosecone shaft 296 can optionally extend through the lumen 212 of the balloon catheter 210 and an internal cavity 220 of the balloon 218 towards the handle 204.

The outer delivery shaft 208, the push shaft 216, the balloon catheter 210, and the nosecone shaft 296, can optionally be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic valve 100 from the outer delivery shaft 208.

The proximal ends of the balloon catheter 210, the outer delivery shaft 208, the push shaft 216, and the nosecone shaft 196, can optionally be coupled to the handle 204. During delivery of the prosthetic valve 100, the handle 204 can optionally be maneuvered by an operator (e.g., a clinician or a surgeon) to axially advance or retract components of the delivery apparatus 202, such as the guidewire 222, the nosecone shaft 296, the tubular body 280, the balloon catheter 210, the outer delivery shaft 208, and/or the push shaft 216, through the patient's vasculature and/or along the target site of implantation, as well as to inflate the balloon 218 mounted on the balloon catheter 210 so as to expand the prosthetic valve 100, and to deflate the balloon 218 and retract the delivery apparatus 202 once the guest prosthetic valve 100 is mounted in the implantation site (e.g., within the host valve).

The balloon catheter 210 can extend through the handle 204 and optionally be fluidly connectable to a fluid source for inflating the balloon 218. The fluid source comprises an inflation fluid. The term “inflation fluid”, as used herein, means a fluid (e.g., saline, though other liquids or gas can be used) used for inflating the balloon 218. An inflation fluid source is in fluid communication with the balloon catheter lumen 212, such as the annular space between the inner surface of balloon catheter 210 and the outer surface of nosecone shaft 296 extending therethrough, such that fluid from the fluid source can optionally flow through the balloon catheter lumen 212, and into the balloon 218 to inflate it.

The handle 204 can optionally include a steering mechanism configured to adjust the curvature of the distal end portion of the delivery apparatus 202. In the illustrated example, the handle 204 can optionally include an adjustment member, such as the illustrated rotatable knob 206a, which in turn is operatively coupled to the proximal end portion of a pull wire. The pull wire can optionally extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can optionally further include an adjustment mechanism including an adjustment member, such as the illustrated rotatable knob 206b. The adjustment mechanism can be configured to adjust the axial position of the push shaft 216 relative to the balloon catheter. The handle can optionally include additional adjustment mechanisms controllable by additional knobs to maneuver additional components of the delivery apparatus 202, such as axial movement of the guidewire 222, axial movement of the nosecone shaft 296, axial movement of the balloon catheter 210, axial movement of a push shaft 216, and/or axial movement of the cutting member 278, relative to other shafts, wires or components of the delivery apparatus 202.

The prosthetic valve 100 can optionally be carried by the delivery apparatus 202 during delivery in a crimped state, and expanded by inflation of balloon 218 to secure it in a native heart valve annulus (such as aortic annulus 24) or against a previously implanted prosthetic valve. In an exemplary implantation procedure, the prosthetic valve 100 can optionally be initially crimped over the balloon catheter 210, proximal to the balloon 218. Because prosthetic valve 100 is crimped at a location different from the location of balloon 218, prosthetic valve 100 can be crimped to a lower profile than would be possible if it was crimped on top of balloon 218. This lower profile permits the clinician to more easily navigate the delivery assembly 200 (including crimped prosthetic valve 100) through a patient's vasculature to the treatment location. The lower profile of the crimped prosthetic valve is particularly helpful when navigating through portions of the patient's vasculature which are particularly narrow, such as the iliac artery.

When reaching the host valve, the delivery apparatus 202 can be utilized to modify at least one host leaflet 10 described above with respect to FIGS. 11A-11D, after which the deflated balloon 218, carrying crimped prosthetic valve 100 thereover, can be advanced to the host valvular structure 12 to expand the guest prosthetic valve 100. Prior to inflation of balloon 218, the push shaft 216 can optionally be advanced distally, allowing its distal end portion to contact and push against the outflow end 106 of prosthetic valve 100, pushing the prosthetic valve 100 distally therewith. The distal end of push shaft 216 is dimensioned to engage with the outflow end 106 of prosthetic valve 100 in a crimped configuration of the valve. In some implementations, the distal end portion of the push shaft 216 can optionally be flared radially outward, to terminate at a wider-diameter that can contact the prosthetic valve 100 in its crimped state. Optionally, push shaft 216 can then be advanced distally, pushing the prosthetic valve 100 therewith, until the crimped prosthetic valve 100 is disposed around the balloon 218, after which the balloon 218 can optionally be inflated to radially expand the prosthetic valve 100. Once the prosthetic valve 100 is expanded to its functional diameter within a native annulus or within a previously implanted host prosthetic valve, the balloon 218 can optionally be deflated and the delivery apparatus 202 can optionally be retrieved from the patient's body.

In some examples, the delivery assembly 200 can optionally be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can optionally be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

Nosecone shaft 296, balloon catheter 210, and optional outer delivery shaft 208, can optionally be formed from any of various suitable materials, such as nylon, braided stainless steel wires, or a polyether block amide (commercially available as Pebax®). In some examples, nosecone shaft 296, balloon catheter 210, and optional outer delivery shaft 208, can optionally have longitudinal sections formed from different materials in order to vary the flexibility of the shafts along their lengths. In some examples, nosecone shaft 296 has an inner liner or layer formed of Teflon® to minimize sliding friction with tubular body 280 of cutting member 278. In some examples, tubular body 280 has an inner liner or layer formed of Teflon® to minimize sliding friction with the guidewire 222.

The nosecone shaft 296 can optionally be sized such that an annular space is formed within balloon catheter lumen 212 between balloon catheter 210 and nosecone shaft 296 along the length of balloon catheter 210. This annular space is optionally in fluid communication with one or more balloon catheter openings 214 exposed to an internal cavity 220 of the balloon 218, which can optionally be in fluid communication with a fluid source (e.g., a syringe or a pump) that can optionally inject an inflation fluid (e.g., saline) into balloon cavity 220. In this way, fluid from the fluid source can optionally flow through balloon catheter lumen 212, and into balloon cavity 220 via balloon catheter opening(s) 214, which serves to inflate the balloon 218 and expand and deploy a prosthetic valve 100 disposed thereon. The pressure of the inflation fluid within balloon cavity 220 may provide the force that allows the balloon 218 to expand a prosthetic valve 100 disposed thereon. Further, the balloon catheter lumen 212 may optionally be configured to withdraw fluid from balloon cavity 220 through balloon catheter opening(s) 214, to deflate the balloon 218.

While balloon catheter 210 is shown to terminate at a proximal end of balloon 218 in FIG. 13, in some examples, balloon catheter 210 can optionally extend farther in the distal direction (examples not illustrated), through a portion or through the entire length of balloon cavity 220, and one or more balloon catheter opening(s) 214 can optionally be formed on the sidewall of balloon catheter 210, exposed laterally to balloon cavity 220.

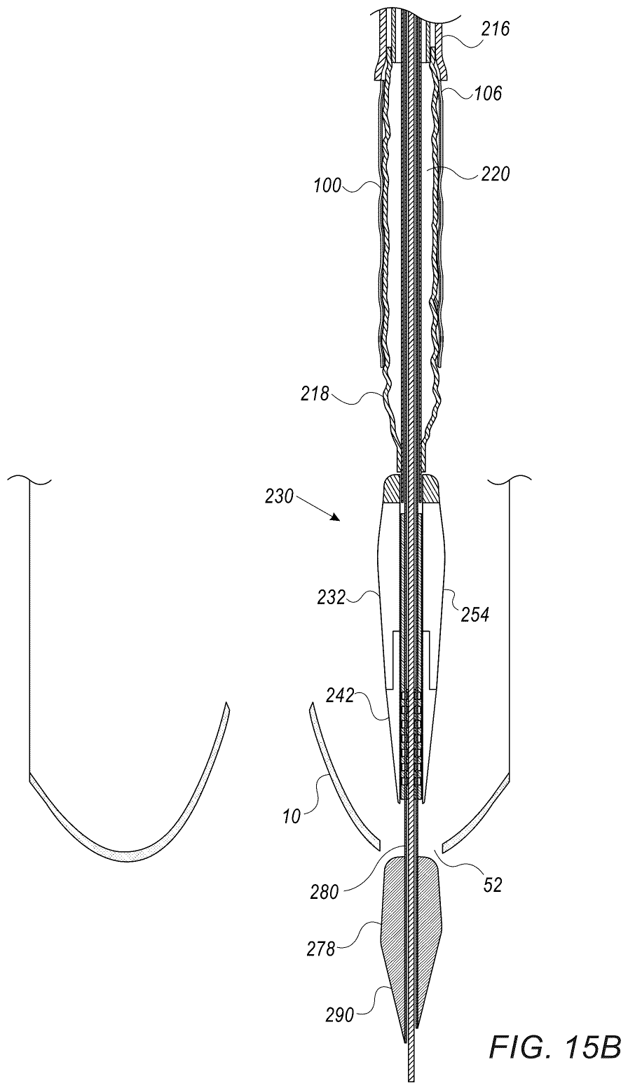

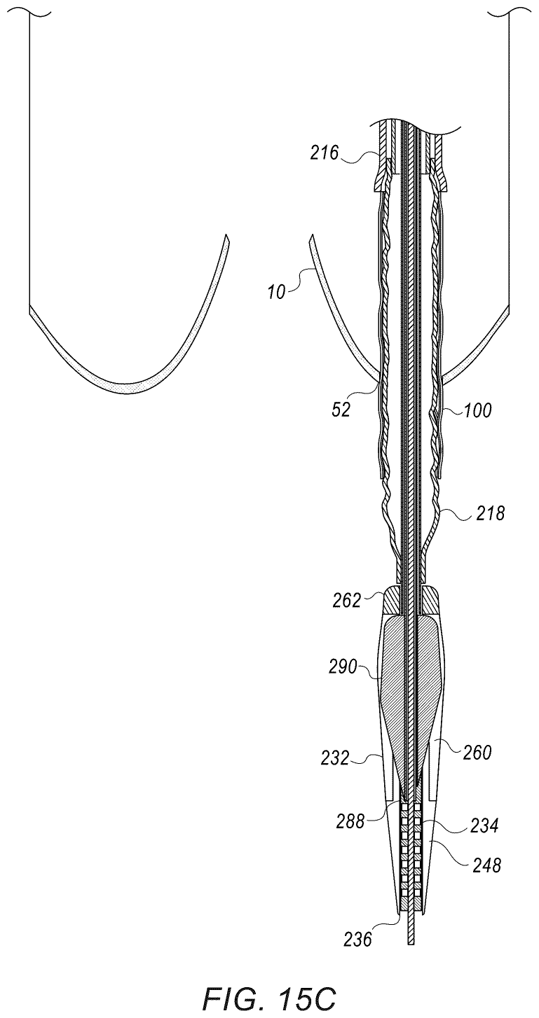

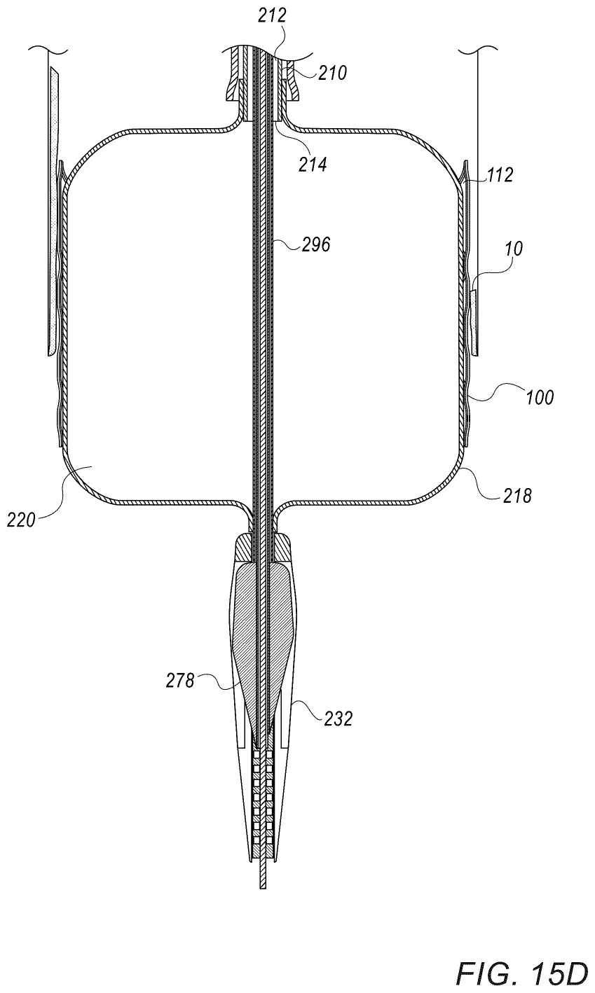

FIGS. 14A-15D show stages in an exemplary implantation procedure for implanting the prosthetic valve 100 within a host valvular structure 12 using a transfemoral delivery procedure. In some examples, various other delivery procedures can optionally be used, such as transventricular, transapical, transseptal, etc. Delivery apparatus 202 may include any of a variety of features to facilitate positioning the guidewire 222, nosecone assembly 232, and/or the cutting member 278, relative to the host leaflet 10. For example, the nosecone shaft 296, balloon catheter 210, outer delivery shaft 208, and/or guidewire 222, may optionally be pre-formed, shaped, and/or curved so as to be directed and/or angled toward the host leaflet 10 when positioned in the vicinity of the host valvular structure 12. Furthermore, one or more shafts of delivery apparatus 202, such as outer delivery shaft 208, can optionally have a steering mechanism (e.g., a pull wire and a corresponding adjustment mechanism in the handle 204) to steer or adjust its distal end.

Formation of a leaflet opening 52 in a host leaflet 10 by a tissue cutting assembly 230 of a delivery assembly 200 can be performed in the same manner described above with respect to FIGS. 11A-11D. FIGS. 14A-14B illustrate the tissue cutting assembly 230 utilized to form a leaflet opening 52, after optionally forming a pilot puncture 50, within the host leaflet 10. In particular, FIG. 14A illustrates an optional step of passing the guidewire 222 through the host leaflet 10, in accordance with examples described above, so as to form a pilot puncture 50 corresponding to the illustrated state shown in FIG. 11A. FIG. 14B illustrates the blade(s) 290 of the cutting member 278 passed through the leaflet 10, optionally following formation of the pilot puncture 50, so as to cut through the tissue and form an enlarged leaflet opening 52. corresponding to the state described above with respect to FIG. 11B. As shown throughout FIGS. 14A-14B, the balloon 218, positioned proximal to the nosecone assembly 232, remains in a deflated state throughout steps of the procedure equivalent to those described above with respect to FIGS. 11A-11D.

FIGS. 15A-15D show optionally subsequent steps of a method utilizing delivery assembly 200, following steps equivalent to those described above and illustrated in FIGS. 11A-11D. After formation of the leaflet opening 52 by cutting member 278 as shown in FIGS. 11C and 15A, the push shaft 216 can optionally be utilized to distally advance the crimped prosthetic valve 100 toward and around balloon 218, as shown in FIG. 15B. While the nosecone assembly 232 is shown to be positioned proximal to the host leaflet 10 in a manner similar to that illustrated in FIG. 11C, it is to be understood that the push shaft 216 can optionally be utilized to distally advance the crimped prosthetic valve 100 toward and around balloon 218 during and/or after passing the nosecone assembly 232 partially or completely through the host leaflet 10, in a manner that can be similar to that described above with respect to FIG. 11D.

Optionally, the deflated balloon 218 and the prosthetic valve 100 disposed thereover can be then advanced and positioned within the leaflet opening 52, as shown in FIG. 15C. The push shaft 216 can optionally remain in position, abutting outflow end 106 of prosthetic valve 100 during advancement into and through the leaflet opening 52, to provide a counter force that resists proximal displacement of the prosthetic valve 100 during insertion into leaflet opening 52.

At this stage, delivering inflation fluid into the balloon cavity 220 allows the balloon 218 to inflate and expand the prosthetic valve 100, as shown in FIG. 15D. As described in more detail below, expanding the guest prosthetic valve 100 to the radially expanded configuration within the leaflet opening 52 of the host leaflet 10 may facilitate preserving access to the coronary arteries 34, 36 and/or maintaining sufficient perfusion of blood to the coronary arteries 34, 36 through the frame 102 of the guest prosthetic valve 100.

While the prosthetic valve 100 is shown in FIG. 15B to be pushed distally to the position of balloon 218 after forming the leaflet opening 52, it is to be understood that the prosthetic valve 100 can optionally be pushed by push shaft 216 over balloon 218 at any other stage prior to advancement of the balloon 218 into the leaflet opening 52 as shown in FIG. 15C. For example, the prosthetic valve 100 can optionally be pushed over balloon 218 upon approximation to the site of implantation, such as upon reaching the region of the ascending aorta 26 even prior to forming the pilot puncture as shown in FIG. 11A. Similarly, the prosthetic valve 100 can optionally be pushed distally after the pilot puncture 50 is formed but before advancing the cutting member 278 into the host leaflet 10, or after passing at least a portion of the nosecone assembly 232 through the leaflet 10, optionally to enlarge leaflet opening 52. Similarly, it is to be understood that in some examples, the delivery assembly 200 can optionally be provided without a push shaft 216, and/or that the prosthetic valve 100 can optionally be crimped around the balloon 218 and delivered through the patient's vasculature in this position.

With the guest prosthetic valve 100 received within the leaflet opening 52, radially expanding the guest prosthetic valve, as shown in FIG. 15D, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell opening 112 of the guest prosthetic valve 100.

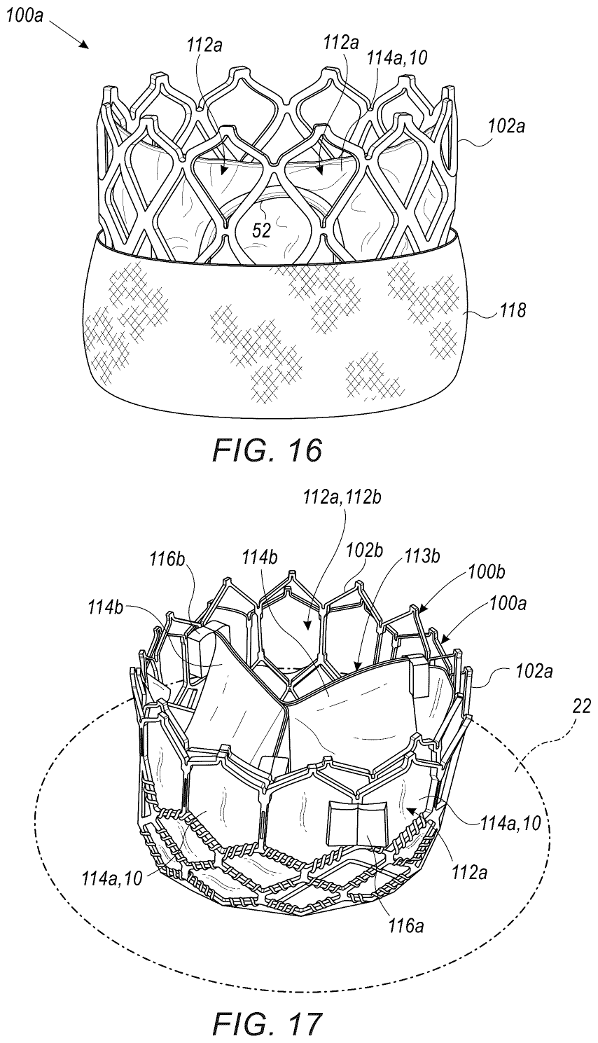

As mentioned, the delivery assemblies and methods of the current specification can optionally be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 16 shows a previously implanted prosthetic valve 100a subsequent to forming the leaflet opening 52, for example subsequent to the method described above with respect to FIGS. 11A-11D. FIG. 17 shows a configuration in which a second prosthetic valve 100b has been expanded within the leaflet opening 52 of a host prosthetic valve 100a. In the example of FIG. 17, the guest prosthetic valve 100b is the same type of valve as the host prosthetic valve 100a. It is to be understood, however, that the methods described herein, when implemented in ViV procedures, also may be applied to any other suitable valvular structures, such as different prosthetic valves and/or native heart valves. For example, the guest prosthetic valve 100b need not be the same type of valve as the host prosthetic valve 100a.

In the example of FIG. 16, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 17, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

While the methods disclosed herein refer to forming a leaflet opening 52 in a host leaflet 10, prior to positioning and expanding a prosthetic valve 100, it is to be understood that any of the methods can optionally comprise, in some examples, repeating one or more steps disclosed throughout the current specification to form a plurality of punctures and openings in the host valvular structure. For example, steps described above with respect to FIGS. 11A-11D can be performed for forming a first leaflet opening in a first host leaflet, after which the delivery apparatus, including tissue cutting assembly 230, can optionally be retracted from the first host leaflet and steered toward another host leaflet, after which the same or equivalent steps can optionally be repeated to form a second leaflet opening within the second host leaflet. The procedure can be optionally repeated to form further leaflet openings, such as a third leaflet opening in a third host leaflet.

In some examples, forming more than one leaflet opening, such as forming the second leaflet opening, can provide further access and/or fluid paths through the frame of the guest prosthetic valve. For example, radially expanding the guest prosthetic valve 100 within the first leaflet opening may push the second host leaflet against the frame of the guest prosthetic valve such that the second leaflet opening is aligned with cell opening(s) of the frame of the guest prosthetic valve. Thus, the second leaflet opening can provide additional unobstructed paths through the frame of the guest prosthetic valve. Moreover, in an example in which the host valve is a previously implanted prosthetic valve, expanding the guest prosthetic valve within the first leaflet opening can trap the second leaflet opening between the respective frames of the host prosthetic valve and the guest prosthetic valve, thereby providing additional access and/or flow paths through each of the frames.

Thus, forming the second leaflet opening can ensure that a greater number of cell openings of the frame are uncovered, and/or that a greater proportion of the frame is uncovered, relative to an example in which only one leaflet is punctured to form a leaflet opening. This may be particularly beneficial in examples in which the frame of a host prosthetic valve extends axially in a downstream direction beyond one or both of the coronary arteries when the guest prosthetic valve is implanted within a native heart valve.

Specifically, in some patient anatomies, the left coronary artery is positioned lower (that is, proximate to the host valvular structure) than the right coronary artery. In such examples, the right coronary artery may be sufficiently far from the host valvular structure that implanting the guest prosthetic heart valve within the host valvular structure does not limit access and/or perfusion to the right coronary artery. Accordingly, forming a single leaflet opening in the host valvular structure may be sufficient to ensure access and/or perfusion to both coronary arteries, provided that the leaflet opening is formed and/or positioned to ensure access to the left coronary artery.

In other examples, however, each of the left and right coronary arteries may be positioned sufficiently proximate to the host valvular structure that forming a single leaflet opening in the host valvular structure is insufficient to ensure access to both coronary arteries. In such examples, forming two leaflet openings in respective leaflets of the previously implanted prosthetic heart valve may ensure the ability for future access into both coronary arteries or perfusion through the frame to both coronary arteries during the diastole phase of the cardiac cycle. In some examples, the host valvular structure can optionally be modified such that the guest prosthetic valve can optionally be implanted by being expanded in a leaflet opening of a first host leaflet that faces the left coronary artery, and such that the second leaflet opening can optionally be formed in a second host leaflet that faces the right coronary artery (or vice-versa).

In some examples, forming the first leaflet opening can optionally be performed prior to forming the second leaflet opening. In other examples, forming the second leaflet opening can optionally be performed prior to forming the first leaflet opening. In some examples, the order of forming leaflet openings is chosen such that the final leaflet opening is formed in the host leaflet in which the guest prosthetic valve 100 is to be positioned and expanded, such as over a balloon as described above with respect to FIGS. 15A-15D.

It is to be understood that the guest prosthetic valve 100 is not limited to being implanted within an opening 52 of a leaflet. For example, in cases where the tissue cutting assembly 230 forms a full tear in a host leaflet that extends to the coaptation edge of the leaflet, the guest prosthetic valve 100 can optionally be positioned at a location between the leaflets of the host valvular structure, for example by retracting the delivery apparatus from the host leaflet in which a leaflet opening is formed, repositioning and readvancing it such that the deflated valve expansion balloon, along with the prosthetic valve 100 disposed thereon, is positioned between the host leaflets, and then inflating the valve expansion balloon to expand the prosthetic valve 100. In some examples, such as in cases where the opening 52 does not form a full tear in the leaflet, the guest prosthetic heart valve can be positioned at a location between the leaflets of the host valvular structure 12 (such that the delivery assembly 200 used to implant to guest prosthetic valve 100 does not extend through the leaflet opening 52) and then expanded. In such cases, the opening 52 may provide sufficient open space through which blood may flow into the coronary ostia, and/or through which additional access devices, such as coronary catheters, can pass during future interventional procedures.

While a delivery assembly 200 comprising a balloon catheter 210 and a balloon-inflatable prosthetic valve 100 that can optionally be expanded by inflating a balloon 218 is described above with respect to FIGS. 12-15D, it is to be understood that utilization of other valve expansion mechanisms in combination with the tissue cutting assembly 230 are contemplated. In some examples, radial expansion of the guest prosthetic valve 100 can optionally be achieved by actuating a mechanical actuator of the guest prosthetic valve to mechanically expand a frame of the guest prosthetic valve. In some examples, the guest prosthetic valve can optionally be a self-expandable prosthetic valve that can optionally be retained during delivery toward the host valvular structure in a capsule or other restraint disposed therearound, and valve expansion can be achieved by removing the capsule or other restraint from the guest prosthetic valve to allow it to radially self-expand within the host valvular structure.

Any of the assemblies, devices, apparatuses, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A tissue cutting assembly comprising: a nosecone assembly defining a nosecone channel, the nosecone assembly comprising: a rigid member comprising at least one rigid member slot; a flexible member positioned distal to the rigid member and attached thereto; and a cutting member comprising a tubular body extending through the nosecone channel, and at least one blade attached to the tubular body, the at least one blade comprising a sharp edge extending radially away from the tubular body; wherein the cutting member is axially movable relative to the nosecone assembly between a concealed position and an exposed position, wherein the at least one blade is entirely concealed within the at least one rigid member slot when the cutting member is in the concealed position, and wherein at least a portion of the at least one blade is exposed out of the nosecone assembly when the cutting member is in the exposed position.

Example 2. The tissue cutting assembly of any example herein, particularly example 1, wherein the nosecone assembly defines a distal tapering portion extending from a nosecone distal edge to a tapering portion proximal end.

Example 3. The tissue cutting assembly of any example herein, particularly example 2, wherein the flexible member comprises the nosecone distal edge.

Example 4. The tissue cutting assembly of any example herein, particularly example 2 or 3, wherein the rigid member comprises the tapering portion proximal end.

Example 5. The tissue cutting assembly any example herein, particularly any one of examples 1 to 4, further comprising a nosecone shaft attached to the nosecone and extending proximally therefrom, the nosecone shaft defining a nosecone shaft lumen sized to allow axial passage of the tubular body therethrough.

Example 6. The tissue cutting assembly any example herein, particularly any one of examples 1 to 5, wherein the rigid member further comprises a rigid member proximal end portion extending proximally from the at least one rigid member slot.

Example 7. The tissue cutting assembly any example herein, particularly any one of examples 1 to 6, wherein the tubular body defines a cutting member lumen sized to allow axial passage of a guidewire therethrough.

Example 8. The tissue cutting assembly any example herein, particularly any one of examples 1 to 7, wherein the sharp edge extends diagonally along an angled edge portion of the at least one blade, wherein the angled edge portion is angularly oriented with respect to the tubular body.

Example 9. The tissue cutting assembly any example herein, particularly any one of examples 1 to 8, wherein the flexible member comprises a flexible member proximal lip, and wherein the rigid member comprises a rigid member distal lip which is in contact with the flexible member proximal lip.

Example 10. The tissue cutting assembly of any example herein, particularly example 9, wherein the rigid member distal lip which is attached to the flexible member proximal lip.

Example 11. The tissue cutting assembly of any example herein, particularly example 9 or 10, wherein the flexible member proximal lip extends radially outward from the nosecone channel.

Example 12. The tissue cutting assembly any example herein, particularly any one of examples 9 to 11, wherein the rigid member distal lip extends radially outward from the nosecone channel.

Example 13. The tissue cutting assembly of any example herein, particularly example 9 or 10, wherein the flexible member comprises a proximal extension extending proximally from the flexible member proximal lip, and wherein the rigid member comprises a socket accommodating the proximal extension therein.

Example 14. The tissue cutting assembly of any example herein, particularly example 13, wherein the proximal extension is attached to the socket.

Example 15. The tissue cutting assembly any example herein, particularly any one of examples 1 to 14, wherein the at least one blade is integrally formed with the tubular body.

Example 16. The tissue cutting assembly any example herein, particularly any one of examples 1 to 15, wherein the tubular body comprises a tube distal end portion configured to pierce through a target tissue.

Example 17. The tissue cutting assembly of any example herein, particularly example 16, wherein the tube distal end portion comprises at least one angled surface terminating with at least one tube tip.

Example 18. The tissue cutting assembly of any example herein, particularly example 17, wherein the sharp edge extends from the tube tip.

Example 19. The tissue cutting assembly any example herein, particularly any one of examples 1 to 18, wherein the flexible member is formed as full-matter surrounding the entire circumference around the nosecone channel.

Example 20. The tissue cutting assembly of any example herein, particularly example 19, wherein the sharp edge of the at least one blade is configured to cut through the flexible member during advancement of the cutting member from the concealed position to the exposed position.

Example 21. The tissue cutting assembly any example herein, particularly any one of examples 1 to 18, wherein the flexible member comprises at least one flexible member slot, extending radially away from the nosecone channel.

Example 22. The tissue cutting assembly of any example herein, particularly example 21, wherein the at least one flexible member slot is aligned with the at least one rigid member slot, together forming a continuous slot through which the at least one blade can axially pass.

Example 23. The tissue cutting assembly of any example herein, particularly example 21 or 22, wherein the at least one flexible member slot extends from the at least one rigid member slot to a distal edge of the flexible member.

Example 24. The tissue cutting assembly any example herein, particularly any one of examples 1 to 18, wherein the flexible member comprises at least one weakened line, configured to be cut by the sharp edge of the at least one blade when the blade is axially passed therethrough.

Example 25. The tissue cutting assembly of any example herein, particularly example 24, wherein the at least one weakened line comprises a thinned line.

Example 26. The tissue cutting assembly of any example herein, particularly example 24, wherein the at least one weakened line comprises a score-line.

Example 27. The tissue cutting assembly of any example herein, particularly example 24, wherein the at least one weakened line comprises at least one slit.

Example 28. The tissue cutting assembly any example herein, particularly any one of examples 24 to 27, wherein the at least one weakened line is aligned with the at least one rigid member slot.

Example 29. The tissue cutting assembly any example herein, particularly any one of examples 1 to 28, wherein the at least one blade comprises a single blade, and wherein the at least one rigid member slot comprises a single rigid member slot.

Example 30. The tissue cutting assembly any example herein, particularly any one of examples 1 to 29, further comprising at least one support member extending axially along at least a portion of the nosecone channel.

Example 31. The tissue cutting assembly of any example herein, particularly example 30, wherein the at least one support member is sized to allow axial passage of the tubular body therethrough.

Example 32. The tissue cutting assembly of any example herein, particularly example 30 or 31, wherein the at least one support member comprises a proximal portion disposed within the rigid member, and a distal portion disposed within the flexible member.

Example 33. The tissue cutting assembly of any example herein, particularly example 32, wherein the distal portion comprises a plurality of windows separated from each other by a plurality of ribs.

Example 34. The tissue cutting assembly of any example herein, particularly example 33, wherein the proximal portion is devoid of windows.

Example 35. The tissue cutting assembly any example herein, particularly any one of examples 30 to 35, wherein the at least one support member comprises a single support member.

Example 36. The tissue cutting assembly of any example herein, particularly example 35, wherein the support member comprises a support member slot.

Example 37. The tissue cutting assembly of any example herein, particularly example 36, wherein the support member slot is aligned with the rigid member slot.

Example 38. The tissue cutting assembly any example herein, particularly any one of examples 1 to 15, wherein the at least one blade comprises two blades.

Example 39. The tissue cutting assembly of any example herein, particularly example 38, wherein the two blades extend in radially opposite directions from the tubular body.

Example 40. The tissue cutting assembly of any example herein, particularly example 38 or 39, wherein the at least one rigid member slot comprises two rigid member slots, each of which is configured to accommodate a respective one of the two blades in the concealed position.

Example 41. The tissue cutting assembly any example herein, particularly any one of examples 38 to 40, wherein the tubular body comprises a tube distal end portion configured to pierce through a target tissue.

Example 42. The tissue cutting assembly of any example herein, particularly example 41, wherein the tube distal end portion comprises two angled surfaces, each angled surface terminating with a separate tube tip.

Example 43. The tissue cutting assembly of any example herein, particularly example 42, wherein the two angled surfaces converge at ends thereof opposite to their respective tube tips.

Example 44. The tissue cutting assembly of any example herein, particularly example 42 or 43, wherein the sharp edge of each of the two blades extends from a respective one of the tube tips.

Example 45. The tissue cutting assembly of any example herein, particularly example 44, wherein the two tube tips are axially offset from each other.

Example 46. The tissue cutting assembly any example herein, particularly any one of examples 38 to 45, further comprising two support members extending axially along at least a portion of the nosecone channel.

Example 47. The tissue cutting assembly of any example herein, particularly example 46, wherein the two support members are configured to allow axial passage of the tubular body therebetween.

Example 48. The tissue cutting assembly of any example herein, particularly example 46 or 47, wherein each of the two support members comprises a proximal portion disposed within the rigid member, and a distal portion disposed within the flexible member.

Example 49. The tissue cutting assembly of any example herein, particularly example 48, wherein each distal portion comprises a plurality of windows separated from each other by a plurality of ribs.

Example 50. The tissue cutting assembly of any example herein, particularly example 49, wherein each of the proximal portions is devoid of windows.

Example 51. The tissue cutting assembly any example herein, particularly any one of examples 46 to 50, wherein the two support members are separated from each other by two gaps.

Example 52. The tissue cutting assembly of any example herein, particularly example 51, wherein each support member is C-shaped.

Example 53. The tissue cutting assembly any example herein, particularly any one of examples 1 to 52, wherein the flexible member is more flexible than the rigid member.

Example 54. The tissue cutting assembly any example herein, particularly any one of examples 1 to 53, wherein the rigid member is formed from a stiffer material than that of the flexible member.

Example 55. A delivery assembly comprising: a guest prosthetic valve comprising a frame movable between a radially compressed and a radially expanded configuration; and a delivery apparatus comprising: a handle; a balloon catheter extending from the handle, the balloon catheter defining a balloon catheter lumen; a balloon mounted on the balloon catheter and in fluid communication with the balloon catheter lumen, the balloon configured to transition between deflated and inflated states thereof; and a tissue cutting assembly comprising: a nosecone assembly defining a nosecone channel, the nosecone assembly comprising: a rigid member comprising at least one rigid member slot; a flexible member positioned distal to the rigid member and attached thereto; and a cutting member comprising a tubular body extending through the nosecone channel, and at least one blade attached to the tubular body, the at least one blade comprising a sharp edge extending radially away from the tubular body; wherein the cutting member is axially movable relative to the nosecone assembly, between a concealed position in which the at least one blade is entirely concealed within the at least one rigid member slot, and an exposed position in which at least a portion of the at least one blade is exposed out of the nosecone assembly.

Example 56. The delivery assembly of any example herein, particularly example 55, wherein the sharp edge of the at least one blade is configured to cut through a host leaflet of a host valvular structure when passed through the host leaflet in the exposed position, so as to form a leaflet opening in the host leaflet.

Example 57. The delivery assembly of any example herein, particularly example 56, wherein, when the guest prosthetic valve is disposed around the balloon and positioned within the host valvular structure, inflation of the balloon expands the guest prosthetic valve to implant the guest prosthetic valve in the host valvular structure.

Example 58. The delivery assembly any example herein, particularly any one of examples 55 to 57, wherein the rigid member is formed from a stiffer material than that of the flexible member.

Example 59. The delivery assembly any example herein, particularly any one of examples 55 to 58, wherein the delivery apparatus further comprises a nosecone shaft attached to the nosecone and extending proximally therefrom, the nosecone shaft defining a nosecone shaft lumen sized to allow axial passage of the tubular body therethrough.

Example 60. The delivery assembly any example herein, particularly any one of examples 55 to 59, wherein the nosecone assembly defines a distal tapering portion extending from a nosecone distal edge to a tapering portion proximal end.

Example 61. The delivery assembly of any example herein, particularly example 60, wherein the flexible member comprises the nosecone distal edge.

Example 62. The delivery assembly of any example herein, particularly example 60 or 61, wherein the rigid member comprises the tapering portion proximal end.

Example 63. The delivery assembly any example herein, particularly any one of examples 55 to 62, wherein the sharp edge extends diagonally along an angled edge portion of the at least one blade, wherein the angled edge portion is angularly oriented with respect to the tubular body.

Example 64. The delivery assembly any example herein, particularly any one of examples 55 to 63, wherein the flexible member comprises a flexible member proximal lip, and wherein the rigid member comprises a rigid member distal lip which is in contact with the flexible member proximal lip.

Example 65. The delivery assembly of any example herein, particularly example 64, wherein the rigid member distal lip which is attached to the flexible member proximal lip.

Example 66. The delivery assembly of any example herein, particularly example 64 or 65, wherein the flexible member proximal lip extends radially outward from the nosecone channel, and wherein the rigid member distal lip extends radially outward from the nosecone channel.

Example 67. The delivery assembly of any example herein, particularly example 64 or 65, wherein the flexible member comprises a proximal extension extending proximally from the flexible member proximal lip, and wherein the rigid member comprises a socket accommodating the proximal extension therein.

Example 68. The delivery assembly of any example herein, particularly example 67, wherein the proximal extension is attached to the socket.

Example 69. The delivery assembly any example herein, particularly any one of examples 55 to 68, wherein the at least one blade is integrally formed with the tubular body.

Example 70. The delivery assembly any example herein, particularly any one of examples 55 to 69, wherein the flexible member is formed as full-matter surrounding the entire circumference around the nosecone channel.

Example 71. The delivery assembly any example herein, particularly any one of examples 55 to 70, wherein the flexible member is devoid of a pre-formed slot extending radially away from the nosecone channel.

Example 72. The delivery assembly of any example herein, particularly example 70 or 71, wherein the sharp edge of the at least one blade is configured to cut through the flexible member during advancement of the cutting member from the concealed position to the exposed position.

Example 73. The delivery assembly any example herein, particularly any one of examples 55 to 69, wherein the flexible member comprises at least one flexible member slot, extending radially away from the nosecone channel.

Example 74. The delivery assembly of any example herein, particularly example 73, wherein the at least one flexible member slot is aligned with the at least one rigid member slot, together forming a continuous slot through which the at least one blade can axially pass.

Example 75. The delivery assembly of any example herein, particularly example 73 or 74, wherein the at least one flexible member slot extends from the at least one rigid member slot to a distal edge of the flexible member.

Example 76. The delivery assembly any example herein, particularly any one of examples 55 to 69, wherein the flexible member comprises at least one weakened line, configured to be cut by the sharp edge of the at least one blade when the blade is axially passed therethrough.

Example 77. The delivery assembly of any example herein, particularly example 76, wherein the at least one weakened line comprises a thinned line.

Example 78. The delivery assembly of any example herein, particularly example 76, wherein the at least one weakened line comprises a score-line.

Example 79. The delivery assembly of any example herein, particularly example 76, wherein the at least one weakened line comprises at least one slit.

Example 80. The delivery assembly any example herein, particularly any one of examples 76 to 79, wherein the at least one weakened line is aligned with the at least one rigid member slot.

Example 81. The delivery assembly any example herein, particularly any one of examples 55 to 80, wherein the at least one blade comprises a single blade, and wherein the at least one rigid member slot comprises a single rigid member slot.

Example 82. The delivery assembly of any example herein, particularly example 81, wherein the tubular body comprises a tube distal end portion that comprises an angled surface terminating with a tube tip.

Example 83. The delivery assembly of any example herein, particularly example 82, wherein the sharp edge extends from the tube tip.

Example 84. The delivery assembly any example herein, particularly any one of examples 81 to 83, wherein the tissue cutting assembly further comprises a support member extending axially along at least a portion of the nosecone channel, wherein the support member comprises a support member slot.

Example 85. The delivery assembly of any example herein, particularly example 84, wherein the support member comprises a distal portion disposed within the flexible member and comprising a plurality of windows separated by ribs, and a support member proximal portion disposed within the rigid member and devoid of windows.

Example 86. The delivery assembly of any example herein, particularly example 84 or 85, wherein the support member is disposed between the tubular body and the nosecone assembly.

Example 87. The delivery assembly any example herein, particularly any one of examples 84 to 86, wherein the support member slot is aligned with the rigid member slot, and is configured to allow passage of the blade therethrough.

Example 88. The delivery assembly any example herein, particularly any one of examples 55 to 80, wherein the at least one blade comprises two blades, and wherein the at least one rigid member slot comprises two rigid member slots, each of the two slots aligned with a respective one of the two blades.

Example 89. The delivery assembly of any example herein, particularly example 88, wherein the two blades extend in radially opposite directions from the tubular body.

Example 90. The delivery assembly of any example herein, particularly example 88 or 89, wherein the tubular body comprises a tube distal end portion comprising a U-shaped angled surface terminating at two tube tips.

Example 91. The delivery assembly of any example herein, particularly example 90, wherein the sharp edge of each of the two blades extends from a respective one of the tube tips.

Example 92. The delivery assembly of any example herein, particularly example 91, wherein the two tube tips are axially offset from each other.

Example 93. The delivery assembly any example herein, particularly any one of examples 88 to 92, wherein the tissue cutting assembly further comprises two C-shaped support members extending axially along at least a portion of the nosecone channel, the two support members separated from each other by two gaps.

Example 94. The delivery assembly of any example herein, particularly example 93, wherein each of the two support members comprises a distal portion disposed within the flexible member and comprising a plurality of windows separated by ribs, and a support member proximal portion disposed within the rigid member and devoid of windows.

Example 95. The delivery assembly of any example herein, particularly example 93 or 94, wherein the two support members are disposed between the tubular body and the nosecone assembly.

Example 96. The delivery assembly any example herein, particularly any one of examples 93 to 95, wherein each gap is aligned with a corresponding one of the two rigid member slots, and is configured to allow passage of the blade therethrough.

Example 97. The delivery assembly any example herein, particularly any one of examples 55 to 96, wherein the flexible member is more flexible than the rigid member.

Example 98. The delivery assembly any example herein, particularly any one of examples 55 to 97, further comprising a guidewire extending through a cutting member lumen defined by the tubular body.

Example 99. The delivery assembly of any example herein, particularly example 56 or 57, further comprising a guidewire extending through a cutting member lumen defined by the tubular body, wherein the guidewire comprises a sharp tip configured to penetrate through the host leaflet.

Example 100. The delivery assembly of any example herein, particularly example 98, further comprising an RF energy source coupled to the guidewire and configured to provide RF energy to a tip of the guidewire.

Example 101. The delivery assembly any example herein, particularly any one of examples 55 to 100, wherein the delivery apparatus further comprises a push shaft configured to push the guest prosthetic valve from a position proximal to the balloon toward the balloon.

Example 102. The delivery assembly of any example herein, particularly example 56 or 57, wherein the host valvular structure is a native valvular structure of native heart valve.

Example 103. The delivery assembly of any example herein, particularly example 56 or 57, wherein the host valvular structure is a valvular structure of previously implanted prosthetic valve that is implanted within a native heart valve.

Example 104. The delivery assembly any example herein, particularly any one of examples 55 to 103, wherein the delivery apparatus is sterilized.

Example 105. A method of forming an opening in a target tissue, the method comprising: advancing a tissue cutting assembly to a target tissue, wherein the tissue cutting assembly comprises: a nosecone assembly defining a nosecone channel, and a cutting member comprising a tubular body extending through the nosecone channel, and at least one blade with a sharp edge which is concealed in at least one rigid member slot of a rigid member of the nosecone assembly, proximal to a flexible member of the nosecone assembly attached to the rigid member; translating the cutting member distally relative to the nosecone assembly, so as to expose the sharp edge of the at least one blade; and forming a tissue opening within the target tissue by axially moving the at least one blade through the target tissue.

Example 106. The method of any example herein, particularly example 105, wherein the translating the cutting member comprises distally pushing the tubular body relative to the nosecone assembly.

Example 107. The method of any example herein, particularly example 105 or 106, wherein the translating the cutting member comprises proximally pulling the nosecone assembly relative to the tubular body.

Example 108. The method any example herein, particularly any one of examples 105 to 107, wherein the flexible member is more flexible than the rigid member.

Example 109. The method any example herein, particularly any one of examples 105 to 108, wherein the rigid member is formed from a stiffer material than that of the flexible member.

Example 110. The method any example herein, particularly any one of examples 105 to 109, wherein the at least one blade extends radially away from the tubular body.

Example 111. The method of any example herein, particularly example 110, wherein the sharp edge extends diagonally along an angled edge portion of the at least one blade, wherein the angled edge portion is angularly oriented with respect to the tubular body.

Example 112. The method any example herein, particularly any one of examples 105 to 111, wherein the translating the cutting member comprises passing the at least one blade through the flexible member in a manner that cuts through the flexible member.

Example 113. The method any example herein, particularly any one of examples 105 to 111, wherein the translating the cutting member comprises passing the at least one blade through at least one flexible member slot which is pre-formed in the flexible member.

Example 114. The method any example herein, particularly any one of examples 105 to 111, wherein the translating the cutting member comprises passing the at least one blade through at least one weakened line of the flexible member in a manner that cuts through the weakened line.

Example 115. The method of any example herein, particularly example 114, wherein the at least one weakened line comprises a thinned line.

Example 116. The method of any example herein, particularly example 114, wherein the at least one weakened line comprises a score-line.

Example 117. The method of any example herein, particularly example 114, wherein the at least one weakened line comprises at least one slit.

Example 118. The method any example herein, particularly any one of examples 105 to 117, wherein the tissue cutting assembly further comprises at least one support member disposed within the nosecone channel, and sandwiched between the tubular body and the nosecone assembly.

Example 119. The method any example herein, particularly any one of examples 105 to 118, wherein the at least one blade comprises a single blade, and wherein the at least one rigid member slot comprises a single rigid member slot.

Example 120. The method any example herein, particularly any one of examples 105 to 118, wherein the at least one blade comprises two blades, and wherein the at least one rigid member slot comprises two rigid member slots, each of the two slots aligned with a respective one of the two blades.

Example 121. The method of any example herein, particularly example 120, wherein the two blades extend in radially opposite directions from the tubular body.

Example 122. The method of any example herein, particularly example 120 or 121, wherein distal tips of the sharp edges of the two blades are axially offset from each other.

Example 123. The method any example herein, particularly any one of examples 105 to 118, further comprising, prior to the forming the tissue opening, forming a pilot puncture within the target tissue.

Example 124. The method of any example herein, particularly example 123, wherein the forming the tissue opening comprises passing the at least one blade through the pilot puncture, such that the at least one blade cuts through the target tissue so as to expand the pilot puncture to form the tissue opening.

Example 125. The method of any example herein, particularly example 123 or 124, wherein the forming the pilot puncture comprises passing a tube distal end portion of the tubular body through the target tissue.

Example 126. The method of any example herein, particularly example 125, wherein the tube distal end portion comprises an angled surface terminating at a tube tip.

Example 127. The method of any example herein, particularly example 125, wherein the tube distal end portion comprises a U-shaped angled surface terminating at two tube tips.

Example 128. The method of any example herein, particularly example 127, wherein the two tube tips are axially offset from each other.

Example 129. The method any example herein, particularly any one of examples 125 to 128, wherein the forming the pilot puncture comprises perforating the target tissue by a guidewire extending through the tubular body, followed by additionally piercing the target tissue by the tube distal end portion, advanced over the guidewire to form the pilot puncture.

Example 130. The method of any example herein, particularly example 123 or 124, wherein the forming the pilot puncture comprises perforating the target tissue by a guidewire extending through the tubular body.

Example 131. The method of any example herein, particularly example 129 or 130, wherein the forming the pilot puncture comprises applying RF energy to a tip of the guidewire.

Example 132. The method any example herein, particularly any one of examples 105 to 131, wherein the nosecone assembly defines a distal tapering portion extending from a nosecone distal edge to a tapering portion proximal end.

Example 133. The method of any example herein, particularly example 132, wherein the flexible member comprises the nosecone distal edge, and wherein the rigid member comprises the tapering portion proximal end.

Example 134. The method of any example herein, particularly example 132 or 133, further comprising, subsequent to the axially moving the at least one blade through the target tissue, advancing the nosecone assembly through the tissue opening to further expand the tissue opening.

Example 135. The method any example herein, particularly any one of examples 105 to 134, wherein the advancing the tissue cutting assembly to the target tissue comprises positioning the nosecone assembly in proximity to the target tissue.

Example 136. The method any example herein, particularly any one of examples 105 to 135, wherein the target tissue is the interatrial septum.

Example 137. The method any example herein, particularly any one of examples 105 to 135, wherein the target tissue is a host leaflet of a host valvular structure, and wherein the tissue opening is a leaflet opening.

Example 138. The method of any example herein, particularly example 137, wherein the host valvular structure is a native valvular structure of native heart valve.

Example 139. The method of any example herein, particularly example 137, wherein the host valvular structure is a valvular structure of previously implanted prosthetic valve that is implanted within a native heart valve.

Example 140. The method any example herein, particularly any one of examples 137 to 139, wherein the forming the leaflet opening comprises forming a first leaflet opening in a first host leaflet of the host valvular structure, and wherein, subsequent to forming the first leaflet opening, the method further comprises: retracting the tissue cutting assembly from the first host leaflet; positioning the tissue cutting assembly in proximity to a second host leaflet of the host valvular structure; and forming a second leaflet opening within the second host leaflet by axially moving the at least one blade through the second host leaflet.

Example 141. The method of any example herein, particularly example 140, further comprising, prior to the positioning the tissue cutting assembly in proximity to the second host leaflet, concealing the at least one blade inside the nosecone assembly.

Example 142. The method of any example herein, particularly example 141, further comprising, prior to forming the second leaflet opening, translating the cutting member distally relative to the nosecone assembly, so as to expose the sharp edge of the at least one blade.

Example 143. The method any example herein, particularly any one of examples 137 to 142, further comprising, subsequent to forming the leaflet opening: positioning a guest prosthetic valve in a radially compressed state within the host valvular structure; and radially expanding the guest prosthetic valve.

Example 144. The method of any example herein, particularly example 143, wherein the positioning the guest prosthetic valve within the host valvular structure comprises positioning the guest prosthetic valve within the leaflet opening.

Example 145. The method of any example herein, particularly example 143 or 144, wherein radially expanding the guest prosthetic valve comprises inflating a balloon over which the guest prosthetic valve is disposed.

Example 146. The method of any example herein, particularly example 143 or 144, wherein radially expanding the guest prosthetic valve comprises actuating a mechanical actuator of the guest prosthetic valve.

Example 147. The method of any example herein, particularly example 143 or 144, wherein the guest prosthetic valve is a self-expandable prosthetic valve, and wherein radially expanding the guest prosthetic valve comprises removing a restraint from around the guest prosthetic valve.

Example 148. The method of any example herein, particularly example 145, further comprising, prior to the inflating the balloon: positioning the balloon in a deflated state thereof, along with the guest prosthetic valve disposed in a compressed state over the balloon, inside the host valvular structure.

Example 149. The method of any example herein, particularly example 148, wherein the positioning the balloon inside the host valvular structure comprises positioning the balloon between host leaflets of the host valvular structure.

Example 150. The method of any example herein, particularly example 148 or 149, further comprising, prior to the positioning the balloon, concealing the at least one blade inside the nosecone assembly.

Example 151. The method any example herein, particularly any one of examples 148 to 150, wherein the positioning the balloon inside the host valvular structure comprises positioning the balloon inside the leaflet opening.

Example 152. The method of any example herein, particularly example 151, wherein the inflating the balloon to radially expand the guest prosthetic valve increases the size of the leaflet opening.

Example 153. The method of any example herein, particularly example 151 or 152, wherein the inflating the balloon tears the host leaflet.

Example 154. The method any example herein, particularly any one of examples 148 to 153, wherein the inflating the balloon modifies the host leaflet such that the host leaflet does not obstruct a cell opening of a frame of the guest prosthetic valve.

Example 155. The method any example herein, particularly any one of examples 148 to 154, wherein the inflating the balloon moves the host leaflet to a location upstream of a downstream edge of an outer skirt of the guest prosthetic valve.

Example 156. The method any example herein, particularly any one of examples 148 to 155, wherein a delivery apparatus comprising the tissue cutting assembly, further comprises a nosecone shaft attached to the nosecone and extending proximally therefrom, the nosecone shaft defining a nosecone shaft lumen sized to allow axial passage of the tubular body therethrough.

Example 157. The method of any example herein, particularly example 156, wherein the nosecone shaft extends through a balloon catheter on which the balloon is mounted.

Example 158. The method any example herein, particularly any one of examples 156 to 157, further comprising, prior to positioning the balloon inside the host valvular structure, distally pushing the guest prosthetic valve, by a push shaft of the delivery apparatus, towards and over the balloon.

Example 159. The method of any example herein, particularly example 158, wherein the positioning the balloon comprises keeping the push shaft in close proximity to a proximal end of the guest prosthetic valve, so as to provide a counterforce to prevent the guest prosthetic valve from proximally slipping from the balloon.

Example 160. The method any example herein, particularly any one of examples 148 to 156, wherein the inflating the balloon comprises providing inflation fluid into the balloon via a lumen of a balloon catheter on which the balloon is mounted.

Example 161. The method any example herein, particularly any one of examples 148 to 160, further comprising, subsequent to inflating the balloon to radially expand the guest prosthetic valve, deflating the balloon and retrieving the balloon and the tissue cutting assembly.

Example 162. A method of implanting a guest prosthetic valve within a host valvular structure, the method comprising: advancing a delivery assembly that comprises a delivery apparatus carrying a guest prosthetic valve in a radially compressed state, to a host valvular structure, wherein the delivery apparatus comprises a balloon mounted on a balloon catheter and a tissue cutting assembly which comprises: a nosecone assembly defining a nosecone channel, and a cutting member comprising a tubular body extending through the nosecone channel, and at least one blade with a sharp edge which is concealed in a rigid member slot of a rigid member of the nosecone assembly, proximal to a flexible member of the nosecone assembly attached to the rigid member; translating the cutting member distally relative to the nosecone assembly, so as to expose the sharp edge of the at least one blade; forming a leaflet opening within a host leaflet of the host valvular structure by axially moving the at least one blade through the host leaflet; positioning the balloon in a deflated state thereof, along with the guest prosthetic valve disposed in a compressed state over the balloon, inside the host valvular structure; and inflating the balloon so as to radially expand the guest prosthetic valve.

Example 163. The method of any example herein, particularly example 162, wherein the advancing a delivery assembly to the host valvular structure comprises positioning the nosecone assembly in proximity to the host leaflet.

Example 164. The method of any example herein, particularly example 162 or 163, wherein the translating the cutting member comprises distally pushing the tubular body relative to the nosecone assembly.

Example 165. The method any example herein, particularly any one of examples 162 to 164, wherein the translating the cutting member comprises proximally pulling the nosecone assembly relative to the tubular body.

Example 166. The method any example herein, particularly any one of examples 162 to 165, wherein the flexible member is more flexible than the rigid member.

Example 167. The method any example herein, particularly any one of examples 162 to 166, wherein the rigid member is formed from a stiffer material than that of the flexible member.

Example 168. The method any example herein, particularly any one of examples 162 to 167, wherein the at least one blade extends radially away from the tubular body.

Example 169. The method of any example herein, particularly example 168, wherein the sharp edge extends diagonally along an angled edge portion of the at least one blade, wherein the angled edge portion is angularly oriented with respect to the tubular body.

Example 170. The method any example herein, particularly any one of examples 162 to 169, wherein the translating the cutting member comprises passing the at least one blade through the flexible member in a manner that cuts through the flexible member.

Example 171. The method any example herein, particularly any one of examples 162 to 169, wherein the translating the cutting member comprises passing the at least one blade through at least one flexible member slot which is pre-formed in the flexible member.

Example 172. The method any example herein, particularly any one of examples 162 to 169, wherein the translating the cutting member comprises passing the at least one blade through at least one weakened line of the flexible member in a manner that cuts through the weakened line.

Example 173. The method of any example herein, particularly example 172, wherein the at least one weakened line comprises a thinned line.

Example 174. The method of any example herein, particularly example 172, wherein the at least one weakened line comprises a score-line.

Example 175. The method of any example herein, particularly example 172, wherein the at least one weakened line comprises at least one slit.

Example 176. The method any example herein, particularly any one of examples 162 to 175, wherein the tissue cutting assembly further comprises at least one support member disposed within the nosecone channel, and sandwiched between the tubular body and the nosecone assembly.

Example 177. The method any example herein, particularly any one of examples 162 to 176, wherein the at least one blade comprises a single blade, and wherein the at least one rigid member slot comprises a single rigid member slot.

Example 178. The method any example herein, particularly any one of examples 162 to 176, wherein the at least one blade comprises two blades, and wherein the at least one rigid member slot comprises two rigid member slots, each of the two slots aligned with a respective one of the two blades.

Example 179. The method of any example herein, particularly example 178, wherein the two blades extend in radially opposite directions from the tubular body.

Example 180. The method of any example herein, particularly example 178 or 179, wherein distal tips of the sharp edges of the two blades are axially offset from each other.

Example 181. The method any example herein, particularly any one of examples 162 to 180, further comprising, prior to the forming the leaflet opening, forming a pilot puncture within the host leaflet.

Example 182. The method of any example herein, particularly example 181, wherein the forming the leaflet opening comprises passing the at least one blade through the pilot puncture, such that the at least one blade cuts through the host leaflet so as to expand the pilot puncture to form the leaflet opening.

Example 183. The method of any example herein, particularly example 181 or 182, wherein the forming the pilot puncture comprises passing a tube distal end portion of the tubular body through the host leaflet.

Example 184. The method of any example herein, particularly example 183, wherein the tube distal end portion comprises an angled surface terminating at a tube tip.

Example 185. The method of any example herein, particularly example 183, wherein the tube distal end portion comprises a U-shaped angled surface terminating at two tube tips.

Example 186. The method of any example herein, particularly example 185, wherein the two tube tips are axially offset from each other.

Example 187. The method any example herein, particularly any one of examples 183 to 186, wherein the forming the pilot puncture comprises perforating the host leaflet by a guidewire extending through the tubular body, followed by additionally piercing the host leaflet by the tube distal end portion, advanced over the guidewire to form the pilot puncture.

Example 188. The method of any example herein, particularly example 181 or 182, wherein the forming the pilot puncture comprises perforating the host leaflet by a guidewire extending through the tubular body.

Example 189. The method of any example herein, particularly example 187 or 188, wherein the forming the pilot puncture comprises applying RF energy to a tip of the guidewire.

Example 190. The method any example herein, particularly any one of examples 162 to 189, wherein the nosecone assembly defines a distal tapering portion extending from a nosecone distal edge to a tapering portion proximal end.

Example 191. The method of any example herein, particularly example 190, wherein the flexible member comprises the nosecone distal edge, and wherein the rigid member comprises the tapering portion proximal end.

Example 192. The method of any example herein, particularly example 190 or 191, further comprising, subsequent to the axially moving the at least one blade through the host leaflet, advancing the nosecone assembly through the leaflet opening to further expand the tissue opening.

Example 193. The method any example herein, particularly any one of examples 162 to 192, wherein the positioning the balloon inside the host valvular structure comprises positioning the balloon between host leaflets of the host valvular structure.

Example 194. The method any example herein, particularly any one of examples 162 to 193, further comprising, prior to the positioning the balloon, concealing the at least one blade inside the nosecone assembly.

Example 195. The method any example herein, particularly any one of examples 162 to 194, wherein the positioning the balloon inside the host valvular structure comprises positioning the balloon inside the leaflet opening.

Example 196. The method of any example herein, particularly example 195, wherein the inflating the balloon to radially expand the guest prosthetic valve increases the size of the leaflet opening.

Example 197. The method of any example herein, particularly example 195 or 196, wherein the inflating the balloon to radially expand the guest prosthetic valve tears the host leaflet.

Example 198. The method any example herein, particularly any one of examples 162 to 197, wherein the inflating the balloon to radially expand the guest prosthetic valve modifies the host leaflet such that the host leaflet does not obstruct a cell opening of a frame of the guest prosthetic valve.

Example 199. The method any example herein, particularly any one of examples 162 to 198, wherein the inflating the balloon to radially expand the guest prosthetic valve moves the host leaflet to a location upstream of a downstream edge of an outer skirt of the guest prosthetic valve.

Example 200. The method any example herein, particularly any one of examples 162 to 198, wherein the delivery apparatus further comprises a nosecone shaft attached to the nosecone and extending proximally therefrom, the nosecone shaft defining a nosecone shaft lumen sized to allow axial passage of the tubular body therethrough.

Example 201. The method of any example herein, particularly example 200, wherein the nosecone shaft extends through the balloon catheter.

Example 202. The method any example herein, particularly any one of examples 162 to 201, further comprising, prior to positioning the balloon inside the host valvular structure, distally pushing the guest prosthetic valve, by a push shaft of the delivery apparatus, towards and over the balloon.

Example 203. The method of any example herein, particularly example 202, wherein the positioning the balloon comprises keeping the push shaft in close proximity to a proximal end of the guest prosthetic valve, so as to provide a counterforce to prevent the guest prosthetic valve from proximally slipping from the balloon.

Example 204. The method any example herein, particularly any one of examples 162 to 203, wherein the inflating the balloon comprises providing inflation fluid into the balloon via a lumen of the balloon catheter.

Example 205. The method any example herein, particularly any one of examples 162 to 204, wherein the host valvular structure is a native valvular structure of native heart valve.

Example 206. The method any example herein, particularly any one of examples 162 to 204, wherein the host valvular structure is a valvular structure of previously implanted prosthetic valve that is implanted within a native heart valve.

Example 207. The method any example herein, particularly any one of examples 162 to 206, wherein the forming the leaflet opening comprises forming a first leaflet opening in a first host leaflet of the host valvular structure, and wherein, subsequent to forming the first leaflet opening, the method further comprises: retracting the tissue cutting assembly from the first host leaflet; positioning the tissue cutting assembly in proximity to a second host leaflet of the host valvular structure; and forming a second leaflet opening within the second host leaflet by axially moving the at least one blade through second host leaflet.

Example 208. The method of any example herein, particularly example 191, further comprising, prior to the positioning the tissue cutting assembly in proximity to the second host leaflet, concealing the at least one blade inside the nosecone assembly.

Example 209. The method of any example herein, particularly example 208, further comprising, prior to forming the second leaflet opening, translating the cutting member distally relative to the nosecone assembly, so as to expose the sharp edge of the at least one blade.

Example 210. The method any example herein, particularly any one of examples 207 to 209, wherein the positioning the balloon inside the host valvular structure comprises positioning the balloon inside the second leaflet opening.

Example 211. The method any example herein, particularly any one of examples 162 to 210, further comprising, subsequent to inflating the balloon to radially expand the guest prosthetic valve, deflating the balloon and retrieving the delivery apparatus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A tissue cutting assembly, comprising:
a nosecone assembly defining a nosecone channel, the nosecone assembly comprising:
a rigid member comprising at least one rigid member slot; and
a flexible member positioned distal to the rigid member and attached thereto; and
a cutting member comprising a tubular body extending through the nosecone channel, and at least one blade attached to the tubular body, the at least one blade comprising a sharp edge extending radially away from the tubular body;
wherein the cutting member is axially movable relative to the nosecone assembly between a concealed position and an exposed position, wherein the at least one blade is entirely concealed within the at least one rigid member slot when the cutting member is in the concealed position, and wherein at least a portion of the at least one blade is exposed out of the nosecone assembly when the cutting member is in the exposed position.

2. The tissue cutting assembly of claim 1, wherein the nosecone assembly defines a distal tapering portion extending from a nosecone distal edge to a tapering portion proximal end.

3. The tissue cutting assembly of claim 1, wherein the flexible member comprises a flexible member proximal lip, and wherein the rigid member comprises a rigid member distal lip which is in contact with the flexible member proximal lip.

4. The tissue cutting assembly of claim 3, wherein the rigid member distal lip which is attached to the flexible member proximal lip.

5. The tissue cutting assembly of claim 1, wherein the tubular body comprises a tube distal end portion configured to pierce through a target tissue.

6. The tissue cutting assembly of claim 1, wherein the flexible member is formed as full-matter surrounding the entire circumference around the nosecone channel.

7. The tissue cutting assembly of claim 6, wherein the sharp edge of the at least one blade is configured to cut through the flexible member during advancement of the cutting member from the concealed position to the exposed position.

8. The tissue cutting assembly of claim 1, wherein the flexible member comprises at least one flexible member slot, extending radially away from the nosecone channel.

9. The tissue cutting assembly of claim 8, wherein the at least one flexible member slot is aligned with the at least one rigid member slot, together forming a continuous slot through which the at least one blade can axially pass.

10. The tissue cutting assembly of claim 1, wherein the flexible member comprises at least one weakened line, configured to be cut by the sharp edge of the at least one blade when the blade is axially passed therethrough.

11. The tissue cutting assembly of claim 1, wherein the at least one blade comprises two blades.

12. The tissue cutting assembly of claim 11, wherein the two blades extend in radially opposite directions from the tubular body.

13. The tissue cutting assembly of claim 1, wherein the flexible member is more flexible than the rigid member.

14. A method of forming an opening in a target tissue, the method comprising:
advancing a tissue cutting assembly to a target tissue, wherein the tissue cutting assembly comprises: a nosecone assembly defining a nosecone channel, and a cutting member comprising a tubular body extending through the nosecone channel, and at least one blade with a sharp edge which is concealed in at least one rigid member slot of a rigid member of the nosecone assembly, proximal to a flexible member of the nosecone assembly attached to the rigid member;
translating the cutting member distally relative to the nosecone assembly, so as to expose the sharp edge of the at least one blade; and
forming a tissue opening within the target tissue by axially moving the at least one blade through the target tissue.

15. The method of claim 14, further comprising, prior to the forming the tissue opening, forming a pilot puncture within the target tissue.

16. The method of claim 15, wherein the forming the tissue opening comprises passing the at least one blade through the pilot puncture, such that the at least one blade cuts through the target tissue so as to expand the pilot puncture to form the tissue opening.

17. The method of claim 14, wherein the nosecone assembly defines a distal tapering portion extending from a nosecone distal edge to a tapering portion proximal end.

18. The method of claim 17, wherein the flexible member comprises the nosecone distal edge, and wherein the rigid member comprises the tapering portion proximal end.

19. The method of claim 17, further comprising, subsequent to the axially moving the at least one blade through the target tissue, advancing the nosecone assembly through the tissue opening to further expand the tissue opening.

20. The method of claim 14, wherein the target tissue is a host leaflet of a host valvular structure, and wherein the tissue opening is a leaflet opening.

21. The method of claim 20, further comprising, subsequent to forming the leaflet opening:
positioning a guest prosthetic valve in a radially compressed state within the host valvular structure; and
radially expanding the guest prosthetic valve.
